SEQUENCE LISTING (39 sequences)

SEQ ID NO: 1 - RNA, 94 nt, Artificial Sequence
  Description of artificial sequence Cytohesin- binding nucleic acid sequence
gggagagacaagcuugggucagggaggguagggauuugguugucccauguucgcagccguagaagagaaagagaaguuaauuaaggauccucag

SEQ ID NO: 2 - RNA, 94 nt, Artificial Sequence
  Description of artificial sequence Cytohesin- binding nucleic acid sequence
gggagagacaagcuugggucggguucgcuaaguuggucuaccccucucgaauauccguuaagaagagaaagagaaguuaauuaaggauccucag

SEQ ID NO: 3 - RNA, 93 nt, Artificial Sequence
  Description of artificial sequence Cytohesin- binding nucleic acid sequence
gggagagacaagcuugggucugcucuccaugggugcucuugaaaggggaagggauguggagaagagaaagagaaguuaauuaaggauccucag

SEQ ID NO: 4 - RNA, 94 nt, Artificial Sequence
  Description of artificial sequence Cytohesin- binding nucleic acid sequence
gggagagacaagcuugggucgauguuggcaggcugucgucacuggugggucuaauucccgagaagagaaagagaaguuaauuaaggauccucag

SEQ ID NO: 5 - RNA, 93 nt, Artificial Sequence
  Description of artificial sequence Cytohesin- binding nucleic acid sequence
gggagagacaagcuugggucugcccgcuuagaaaucccaaagugcgggaacgguugcgaagaagagaaagagaaguuaauuaaggauccucag

SEQ ID NO: 6 - RNA, 95 nt, Artificial Sequence
  Description of artificial sequence Cytohesin- binding nucleic acid sequence
gggagagacaagcuugggucgggaaggguggaagugcugcuuggugcucuguccuuuuuuaagaagagaaagagaaguuaauuaaggauccucag

SEQ ID NO: 7 - RNA, 96 nt, Artificial Sequence
  Description of artificial sequence Cytohesin- binding nucleic acid sequence
gggagagacaagcuugggucgucauccguguuuugucauguuuugaugggguuuggggugugauaagagaaagagaaguuaauuaaggauccucag

SEQ ID NO: 8 - RNA, 94 nt, Artificial Sequence
  Description of artificial sequence Cytohesin- binding nucleic acid sequence
gggagagacaagcuugggucuguuguuggaccaguguuauuuuacgcgauugugcgguugugaagagaaagagaaguuaauuaaggauccucag

SEQ ID NO: 9 - RNA, 94 nt, Artificial Sequence
  Description of artificial sequence Cytohesin- binding nucleic acid sequence
gggagagacaagcuugggucuauuaugccuuuagcuagcgcauucugugggguggguggaagaagagaaagagaaguuaauuaaggauccucag

SEQ ID NO: 10 - RNA, 94 nt, Artificial Sequence
  Description of artificial sequence Cytohesin- binding nucleic acid sequence
gggagagacaagcuugggucugguuugaacgggggcgcaucagccaugcuauaaacuccaagaagagaaagagaaguuaauuaaggauccucag

SEQ ID NO: 11 - RNA, 94 nt, Artificial Sequence
  Description of artificial sequence Randomized nucleic acid sequence
gggagagacaagcuugggucgugucuguguagaggacagggcgagagucugauugucugcagaagagaaagagaaguuaauuaaggauccucag

SEQ ID NO: 12 - DNA, 39 bp, Artificial Sequence
  Description of artificial sequence Primer
tctaatacgactcactatagggagagacaagcttgggtc

SEQ ID NO: 13 - DNA, 30 bp, Artificial Sequence
  Description of artificial sequence Primer
ctgaggatccttaattaacttctctttctc

SEQ ID NO: 14 - DNA, 99 bp, Artificial Sequence
  Description of artificial sequence Primer
ctatagggagagacaagcttgggtcnnnnnnnnnnnnnnnnnnnnnnnnnnnnnnnnnnnnnnnnagaagagaaagagaagttaattaaggatcctcag

SEQ ID NO: 15 - DNA, 80 bp, Artificial Sequence
  Pool N30 Ellington
gggataggatccacatctacgtattannnnnnnnnnnnnnnnnnnnnnnnnnnnnnttcactgcagacttgacgaagctt

SEQ ID NO: 16 - DNA, 40 bp, Artificial Sequence
  Primer 5′-N30-41
gataatacgactcactatagggataggatccacatctacg

SEQ ID NO: 17 - DNA, 24 bp, Artificial Sequence
  Primer 3′-N30-24
aagcttcgtcaagtctgcagtgaa

SEQ ID NO: 18 - DNA, 30 bp, Artificial Sequence
  Aptamer NC1F-C11
ccaccagggataaggtcgaaccctgtcgtc

SEQ ID NO: 19 - DNA, 30 bp, Artificial Sequence; identical to SEQ ID NO: 18
  Aptamer NC1F-C14

SEQ ID NO: 20 - DNA, 30 bp, Artificial Sequence; identical to SEQ ID NO: 18
  Aptamer NC1F-C16

SEQ ID NO: 21 - DNA, 30 bp, Artificial Sequence; identical to SEQ ID NO: 18
  Aptamer NC1F-C17

SEQ ID NO: 22 - DNA, 30 bp, Artificial Sequence; identical to SEQ ID NO: 18
  Aptamer NC1F-C18

SEQ ID NO: 23 - DNA, 30 bp, Artificial Sequence; identical to SEQ ID NO: 18
  Aptamer NC1F-C20

SEQ ID NO: 24 - DNA, 30 bp, Artificial Sequence; identical to SEQ ID NO: 18
  Aptamer NC1F-C21

SEQ ID NO: 25 - DNA, 30 bp, Artificial Sequence; identical to SEQ ID NO: 18
  Aptamer NC1F-C22

SEQ ID NO: 26 - DNA, 30 bp, Artificial Sequence; identical to SEQ ID NO: 18
  Aptamer NC1F-C23

SEQ ID NO: 27 - DNA, 30 bp, Artificial Sequence; identical to SEQ ID NO: 18
  Aptamer NC1F-C25

SEQ ID NO: 28 - DNA, 30 bp, Artificial Sequence; identical to SEQ ID NO: 18
  Aptamer NC1F-C4

SEQ ID NO: 29 - DNA, 30 bp, Artificial Sequence; identical to SEQ ID NO: 18
  Aptamer NC1F-C9

SEQ ID NO: 30 - DNA, 29 bp, Artificial Sequence
  Aptamer NC1F-C10
atacagtataaccggcatgccgcatcgac

SEQ ID NO: 31 - DNA, 30 bp, Artificial Sequence
  Aptamer NC1F-C15
atacacgattccaggccgggaaacgaggac

SEQ ID NO: 32 - DNA, 30 bp, Artificial Sequence
  Aptamer NC1F-C19
ataccataacaccgccggcctgtgtacgcg

SEQ ID NO: 33 - DNA, 30 bp, Artificial Sequence
  Aptamer NC1F-C2
atacaccagcgcgccgagactaactccgac

SEQ ID NO: 34 - DNA, 29 bp, Artificial Sequence
  Aptamer NC1F-C3
ccgaccggccatattacgcgtaatctacc

SEQ ID NO: 35 - DNA, 30 bp, Artificial Sequence
  Aptamer NC1F-C1
cgggcccacgcgcgaccgacctcctaacca

SEQ ID NO: 36 - DNA, 30 bp, Artificial Sequence
  Aptamer NC1F-C12
accccccactcgacaacccaacgcccgcta

SEQ ID NO: 37 - DNA, 29 bp, Artificial Sequence
  Aptamer NC1F-C13
atgcatgcctacgacgcccgccggtcacg

SEQ ID NO: 38 - DNA, 30 bp, Artificial Sequence
  Aptamer NC1F-C7
gcgctgatacatcgacctcgcctaacaagg

SEQ ID NO: 39 - DNA, 30 bp, Artificial Sequence
  Aptamer NC1F-C8
ggccggcaatgcgtaaacagggcccagaag

(19) United States
(12) Patent Application Publication
(10) Pub. No.: US 2004/0170990 A1
(43) Pub. Date: Sep. 2, 2004

(54) Intracellular nucleic acid inhibitors of small guanine nucleotide exchange factors

(76) Inventors: Kolanus Waldemar, Bonn (DE); Michael Famulok, Bonn (DE); Michael Blind, Munchen (DE); Gunter Mayer, Munchen (DE)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/451,493

(22) PCT Filed: Dec. 29, 2001
(86) PCT No.: PCT/EP01/15377

(30) Foreign Application Priority Data
Dec. 30, 2000 (EP) 00128766.3

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The invention relates to a nucleic acid, especially an isolated nucleic acid, that is capable of binding to a guanine nucleotide exchange factor for ADP ribosylation factors, and to the derivatives thereof.

The present invention relates to nucleic acids which are capable of binding to a guanine exchange factor, vectors containing these, cells containing these, uses of the same, compositions containing these, inhibitors for a guanine exchange factor and methods for screening compounds which inhibit the interaction between a guanine nucleotide exchange factor and a nucleic acid.

Proteins of the family of guanine nucleotide exchange factors (ARF-GEF's) for human ADP ribosylation factors (ARF's), herein also designated as ARF-GEF's, wherein the ARF's are in turn small Ras-like GTPases, play a critical role in the transport of intracellular vesicles and the organisation of the actin cytoskeleton. ARF's belong to a large family of monomeric G proteins which participate in a plurality of signal transduction cascades and regulatory process. The ARF proteins of mammals can be divided into three classes: class I (ARF 1, 2, 3), class II (ARF 4 and 5) and class III (ARF 6). ARF's interact with a number of regulatory proteins (ARF-GEF's, GTPase-activating proteins (GAPs), arfaptins), effector proteins (coatomers, phospholipase D, βγ-subunits of G proteins), choleratoxin, and phospholipids. The activity of the ARF proteins is determined by the type of bound nucleotide cofactor. GEF's which promote the release of bound GDP and thus the binding of GTP, activate the ARF proteins. Thus, the exchange of highly affine-bound GDP for GTP is required to activate the ADP ribolysation factors (ARF's) (Bourne et al., Nature 349 (1991), 117-127). The interaction of guanine nucleotide exchange factors (GEF's) with the ARF/GDP complex lowers the affinity for GDP and thus facilitates its dissociation (Jackson. and Casanova, Trends Cell. Biol. 10 (2000), 60-67). Thereafter GTP binds to the free nucleotide binding site of the ARF/GEF complex whereby a conformational change is induced which leads to dissociation of the GEF's and activates the ARF/GTP complex (Peyroche et al., Mol. Cell 3 (1999), 275-285).

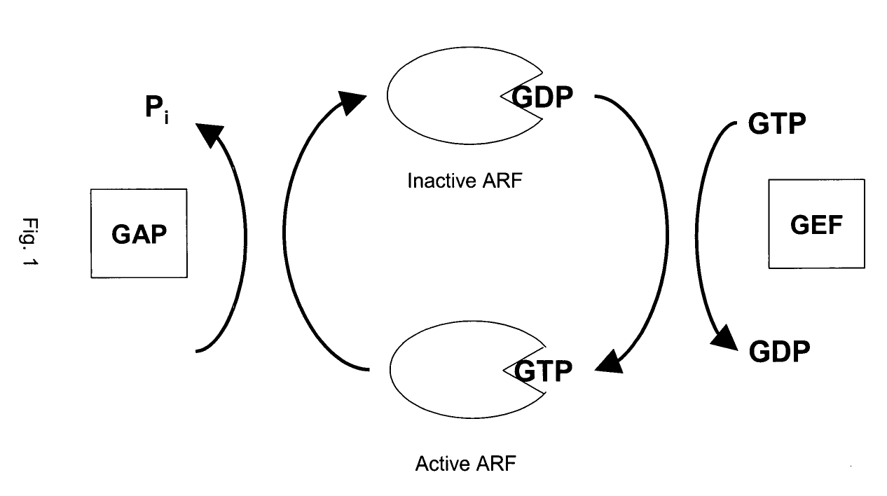

The inactivation of the ARF proteins takes place as a result of the hydrolysis of the bound GTP to GDP, which is accelerated by the GTPase-activating proteins (GAPs). A schematic diagram of this activation/deactivation cycle is shown in FIG. 1. ARF-GEF's can be divided into two families. The large or high-molecular guanine nucleotide exchange factors (ARF-GEF's) having a molecular weight greater than 100 kDa, among others the Sec7 protein from the yeast, Gea1, Gea2, BIG1 or BIG2 are distinguished by their characteristic sensitivity to the low-molecular organic inhibitor Brefeldin A. Brefeldin A binds to the trimeric intermediate complex of ARF, GDP and the large ARF-GEF's, whereby this is stabilised. As a result, the exchange of GDP for GTP is inhibited and the activation of the ARF proteins is prevented. The large ARF-GEF's play a role in regulating the endoplasmic reticulum and the Golgi apparatus. The addition of Brefeldin A leads to breakdown of the Golgi and inhibition of the secretion by the cells. Consequently, Brefeldin A has proved to be a useful substance for studying the function of the large GEF's in vitro and in vivo.

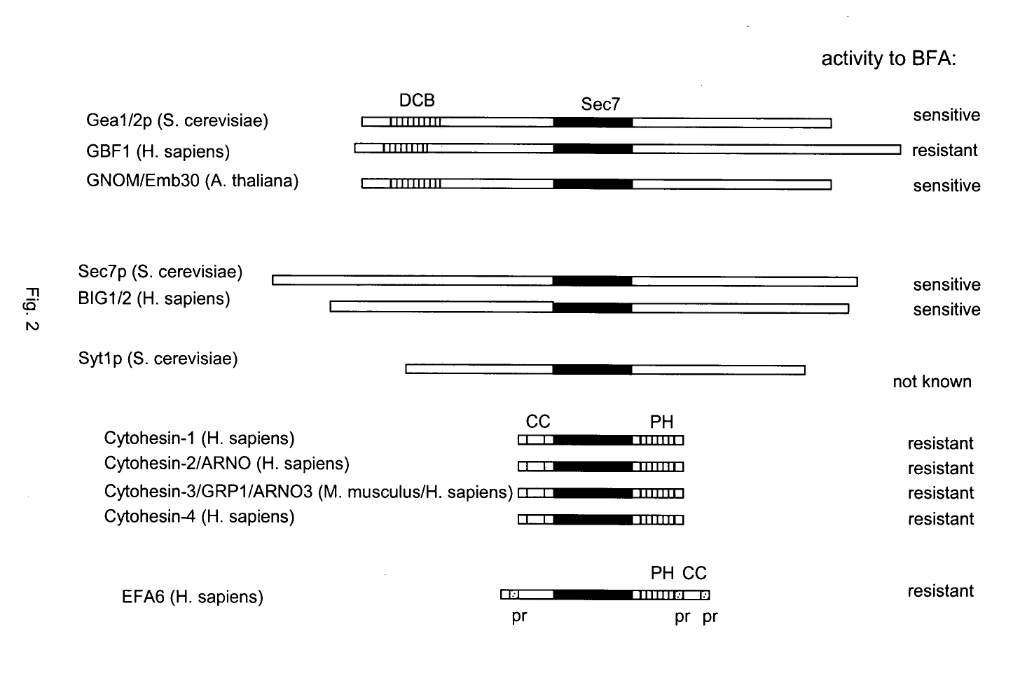

The second group which includes cytohesin-1, cytohesin-2 (ARNO), cytohesin-3 (GRP-1/ARNO3) and cytohesin-4 has a molecular weight of around 50 kDa and is not inhibited by Brefeldin A. The small ARF-GEF's are primarily involved in regulatory processes at the endosomal compartment and at the cytoplasmic membrane. The ARF-GEF family was described among others by Jackson and Casanova (Trends Cell. Biol. 10 (2000), 60-67) and shown in an overview in FIG. 2 (from Donaldson and Jackson, Curr. Opin. Struct. Biol. 12 (2000), 475-482). A common feature of the ARF-GEF's is their central Sec7 domain which is largely responsible for the catalysis of the guanine nucleotide exchange and the sensitivity of the large GEF's to Brefeldin A.

The small ARF-GEF's are implicated in signal transduction processes mediated via PI 3 kinases (Klarlund et al., Science 275 (1997), 1927-1930). Furthermore, they are involved in the reorganisation of the actin cytoskeleton (Frank et al., Mol. Biol. Cell. 9 (1998), 3133-3146) and the activation of integrins (Geiger et al., EMBO J. 16 (2000), 3525-2536). For the small ARF-GEF's no functional modulators or inhibitors have been described hitherto which make it possible to investigate the proteins directly in vitro and in cells. Thus, most results on their biological function have hitherto been obtained by mutation analysis and the expression/overexpression of the proteins or their individual domains in cell culture. All the small ARF-GEF's identified so far contain, in addition to the SEC7 domain, a PH (“pleckstrin homology”) domain which can control the localisation on membranes by interaction with polyphosphoinsitides. In addition, a CC (“coiled coil”) domain was also found which presumably mediates the interaction with other proteins (see FIG. 2 from Donaldson and Jackson, Curr. Opin. Struct. Biol. 12 (2000), 475-482)

The object of the present invention is to provide a modulator for the activity of a guanine nucleotide exchange factor (ARF-GEF) for ARF proteins, especially a small guanine nucleotide exchange factor for ARF proteins. It is furthermore the object of the present invention to provide a modulator for the GDP/GTP exchange function of a guanine nucleotide exchange factor, especially a small guanine nucleotide exchange factor. Finally, it is a further object of the present invention to provide a means and a method for manufacturing such means which intervenes in the process of adhesion of immune cells mediated by integrins of the type β-2, especially leukocytes and thus represents a means which can intervene in the therapy and diagnosis of diseases in which this process is involved.

The object is solved according to the invention by a nucleic acid, especially an isolated nucleic acid which is capable of binding to a guanine nucleotide exchange factor for ADP ribosylation factors and its derivatives.

In a second aspect the object is solved by a nucleic acid which is capable of binding to a guanine nucleotide exchange factor for ADP ribolysation factors, wherein the nucleic acid comprises a sequence selected from the group comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38 and SEQ ID No. 39 as well as their respective derivatives.

In one embodiment of the nucleic acids according to the invention it is provided that the derivatives of the nucleic acid comprises at least one modified compound group, preferably at least one modified compound group in the sugar phosphate backbone and/or at least one modified sugar residue and/or at least one modified base or one or a plurality of combinations thereof.

In one preferred embodiment of the nucleic acids according to the invention it is provided that the modified base is selected from the group comprising 5-fluorouracil, 5-bromouracil, 5-chlorouracil, 5-iodouracil, hypoxanthine, xanthine, ethenoadenosine, 4-acetylcytosine, 5-(carboxyhydroxylmethyl)uracil, 5-carboxymethylaminomethyl-2-thio-uridine, 5-carboxymethylaminomethyluracil, dihydrouracil, β-D-galactosylqueuosine, inosine, N6-isopentenyladenine, 1-methyladenine, 1-methylpseudouracil, 1-methylguanine, 1-methylinosine, 2,2-dimethylguanine, 2-methyladenine, 2-ethylguanine, 3-methylcytosine, 5-methylcytosine, N6-methyladenine, 7-methylguanine, 5-methylaminomethyluracil, 5-methoxyaminomethyl-2-thiouracil, β-D-mannosylqueuosine, 5′-methoxycarbonylmethyluracil, 5-methoxyuracil, 2-methylthio-N6-isopentenyladenine, uracil-5-oxyacetic acid methyl ester, uracil-5-oxyacetic acid, pseudouracil, queuosine, 3-thiocytosine, 5-methyl-2-thiouracil, 2-thiouracil, 4-thiouracil, 5-methyluracil, uracil-5-oxyacetic acid methyl ester, uracil-5-oxyacetic acid, 3(3-amino-3-N-2-carboxypropyl)uracil and 2,6-diaminopurine.

In another embodiment of the nucleic acids according to the invention it is provided that these contain at least one modification or labelling at the 5′ and/or 3′ end wherein the modification and/or labelling is preferably selected from the group containing the biotin group; the digoxigenin group; fluorescent dyes, especially fluorescein and rhodamine; psoralen; thiol group(s), amino group(s), ethylene glycol group(s) or cholesteryl group(s).

In yet another embodiment of the nucleic acids according to the invention it is provided that the modified compound group is selected from the group comprising phosphomono- or phosphodithioates, alkyl phosphonates, aryl phosphonates, phosphoroamidates, phosphate triesters, preferably P(O)-alkyl derivatives; compound groups in which oxygen atoms in the bridge between the sugars formed by the phosphate group are replaced by other bonds, for example NH—, CH₂— or S—P-compounds, more preferably 3′-NHP(O)—(O⁻)O-5′phosphoramidates; dephospho internucleotide compounds, preferably acetamidate, carbamate compounds or peptide nucleic acids (PNA).

In one emdodiment of the nucleic acids according to the invention it is provided that the modified sugar residue is selected from the group comprising 2′-azido-2′-deoxy-, 2′-amino-2′-deoxy-, 2′-fluoro-2′-deoxy-, 2′-chloro-2′-deoxy, 2′-O-methyl, 2′-O-allyl-, 2′-C-fluoromethyl groups and/or modifications.

In one embodiment of the nucleic acids according to the invention it is provided that the guanine nucleotide exchange factor belongs to the group of small guanine nucleotide exchange factors for ARF proteins, especially to the group of guanine nucleotide exchange factors whose molecular weight is around 50 kDa or less.

In yet another embodiment of the nucleic acids according to the invention it is provided that the guanine nucleotide exchange factor is not inhibited by Brefeldin A.

In a preferred embodiment of the nucleic acids according to the invention it is provided that the guanine nucleotide exchange factor is cytohesin-1 or cytohesin-2 and especially the Sec7 domain of cytohesin-1 or cytohesin-2.

In another preferred embodiment of the nucleic acids according to the invention it is provided that that the guanine nucleotide exchange factor is the Sec7 domain.

In yet another embodiment of the nucleic acids according to the invention it is provided that the nucleic acid is such a one selected from the group comprising DNA, RNA, polynucleotides, oligonucleotides, aptamers, aptazymes and intramers.

In a third aspect the object is solved by a vector comprising a nucleic acid according to the invention.

In one embodiment it is provided that the vector is an expression vector.

In a fourth aspect the invention relates to a cell comprising a nucleic acid according to the invention and/or a vector according to the invention.

In one embodiment it is provided that the cell is a eukaryotic cell, preferably an animal cell and quite preferably a mammalian cell.

In another embodiment it is provided that the cell is selected from the group comprising Saccharomyces cerevisiae and C. elegans.

In yet another embodiment of the cell according to the invention it is provided that the mammal is selected from the group comprising mice, rats, rabbits, dogs, pigs, monkeys and humans.

In a fifth aspect the invention relates to an animal, preferably a transgenic animal comprising at least one cell according to the invention.

In one embodiment it is provided that the animal is selected from the group comprising threadworms, fish, mice, rats, rabbits, dogs, pigs and monkeys.

In a sixth aspect the invention relates to the use of a nucleic acid according to the invention for the manufacture of a medicament.

In one embodiment it is provided that the medicament is for the treatment of diseases selected from the group comprising the metastasis of lymphomas or melanomas, autoimmune diseases, rejection reactions, acute and chronic inflammations, refusion damage, transplantation diseases, especially rejection reactions in organ transplantations and graft-vs-host diseases.

In one embodiment of the nucleic acids according to the invention it is provided that the medicament influences the β-2-integrin-mediated adhesion of immune cells.

In a seventh aspect the invention relates to the use of a nucleic acid according to the invention and/or a vector according to the invention in gene therapy.

In an eighth aspect the invention relates to the use of a nucleic acid according to the invention for detection of a guanine nucleotide exchange factor.

In a ninth aspect the invention relates to the use of a nucleic acid according to the invention for complex formation with a guanine nucleotide exchange factor.

In a tenth aspect the invention relates to a composition, especially a pharmaceutical composition, comprising a nucleic acid according to the invention, a vector according to the invention and/or a cell according to the invention together with a suitable support material, especially a pharmaceutically acceptable support.

In an eleventh aspect the invention relates to an inhibitor for a guanine nucleotide exchange factor, wherein the guanine nucleotide exchange factor belongs to the group of small guanine nucleotide exchange factors for ARF proteins, especially to the group of guanine nucleotide exchange factors whose molecular weight is around 50 kDa or less.

In one embodiment it is provided that the guanine nucleotide exchange factor is not inhibited by Brefeldin A.

In another embodiment it is provided the inhibitor comprises a nucleic acid according to the invention and/or a vector according to the invention.

In a twelfth aspect the invention relates to a method for screening compounds which inhibit the interaction between a guanine nucleotide exchange factor and a nucleic according to the invention, especially by a method that is compatible with high-throughput methods and is characterised by the following steps:

a) preparation of the guanine nucleotide exchange factor and the nucleic acid

b) optionally determining whether an interaction takes place between the guanine nucleotide exchange factor and the β-2-integrin,

c) adding a candidate compound and

d) determining whether an interaction between the guanine nucleotide exchange factor and the nucleic acid is inhibited, preferably by the candidate compound.

In one embodiment it is provided that a further step involves determining whether the identified compound inhibits the guanine nucleotide exchange function of the guanine nucleotide exchange factor for a monomeric G protein.

In yet another embodiment it is provided that a further step involves determining whether the interaction of a guanine nucleotide exchange factor with an integrin, preferably the interaction between the Sec-7 domain of cytohesin-1 and the β-2-integrin subunit is inhibited.

In an alternative embodiment it is provided that a further step involves determining whether the interaction of the PH domain with its natural ligands is inhibited, preferably whether the interaction of the PH domain of cytohesin-1 with phosphatidylinositol-3,4,5-trisphosphate is inhibited.

In another embodiment of the method according to the invention it is provided that the candidate substance is used to manufacture a medicament.

In a preferred embodiment it is provided that the medicament is for the treatment of diseases selected from the group comprising the metastasis of lymphomas or melanomas, autoimmune diseases, rejection reactions, acute and chronic inflammations, reperfusion injury, transplantation diseases, especially rejection reactions in organ transplantations and graft-vs-host diseases.

In another embodiment it is provided that the nucleic acid according to the invention, the guanine nucleotide exchange factor or both are labelled with a fluorescent dye and the inhibition of the interaction between the nucleic acid and the guanine nucleotide exchange factor leads to a measurable change in the fluorescence signal.

In yet another embodiment it is provided that the nucleic acid or the guanine nucleotide exchange factor as interaction partner is labelled with a fluorescence-quenching group (quencher), the respective other interaction partner is labelled with a fluorophor group (donor), wherein the interaction between the two interaction partners leads to quenching of the fluorescence emission of the donor and the inhibition of the interaction leads to the release of the fluorescence of the donor.

In one embodiment it is provided that the nucleic acid is an aptazyme and the inhibition of the binding of the guanine nucleotide exchange factor to the aptazyme can be measured by a change in the catalytic activity of the aptazyme.

In yet another embodiment it is provided that the interaction between the interaction partners comprising nucleic acid according to the invention and guanine nucleotide exchange factor takes place by means of detection methods such as enzyme-based assays surface plasmon resonance (SRP), fluorescence activated cell sorting FACS, fibre-optic microarray sensors, capillary electrophoresis, quartz crystal microbalance (QCM) or radioactive measuring methods.

In a thirteenth aspect the invention relates to the use of a guanine nucleotide exchange factor as a target molecule as part of an in vitro selection process.

In a fourteenth aspect the invention relates to a method for identifying and isolating nucleic acids capable of binding to a target molecule, characterised by the following steps:

incubating the target molecule or a part thereof with a plurality of nucleic acids, preferably a nucleic acid library, wherein the nucleic acids have different sequences,

selecting and isolating those nucleic acids capable of binding to the target molecule or a part thereof,

optionally amplifying the isolated nucleic acids and repeating the first two steps; and

optionally determining the sequence and/or binding specificity of the isolated nucleic acids,

wherein the target molecule is a guanine nucleotide exchange factor, preferably one such for ARF.

In a fifteenth aspect the invention relates to the use of a nucleic acid according to the invention, wherein the nucleic acid is preferably present in a complex with a guanine nucleotide exchange factor, for the rational design of compounds. The compounds preferably comprise inhibitors and more preferably low-molecular inhibitors. In connection therewith the structure of the nucleic acids according to the invention serves as a lead structure for low-molecular or small compounds which (can) have an inhibiting effect like the nucleic acids according to the invention.

In the method according to the invention for screening compounds which inhibit the interaction between a guanine nucleotide exchange factor and a nucleic acid according to the invention, it can be provided that the candidate compound is an arbitrary compound which originates from a compound library for example, whose capability should be tested as to whether it is capable of influencing said interaction, especially of inhibiting said interaction.

In the method according to the invention for screening compounds which inhibit the interaction between a guanine nucleotide exchange factor and a nucleic acid according to the invention the detection method can be based on enzyme-based assays similar to antibody-based ELISAs.

In the use according to the invention of a guanine nucleotide exchange factor as target molecule as part of an in vitro selection method it is provided that the ARF-GEF is any ARF-GEF to which the nucleic acids according to the invention bind.

The invention is based on the surprising finding that nucleic acids or nucleic acid ligands can be produced against the small ARF-GEF proteins which in the complex with their respective target, i.e., a guanine nucleotide exchange factor or a part, especially a domain thereof, modulate one or a plurality of functions or activities. The modulation of the function or the activity typically involves an inhibition. The respective function or activity is in this connection typically localised in one or a plurality of domains constructing the guanine exchange factor. An example of an activity which is inhibited by the nucleic acids according to the invention is the GDP/GTP exchange activity of the guanine nucleotide exchange factor. The guanine nucleotide exchange factor is preferably such a one for human ADP ribosylation factors.

The nucleic acids according to the invention are preferably isolated nucleic acids. The concept of nucleic acids should herein also be understood as oligonucleotides and polynucleotides. The concept of isolated nucleic acids should herein especially describe those nucleic acids which are substantially free from nucleic acids having a different nucleic acid sequence. Such isolated nucleic acids are, for example, those produced by chemical or enzymatic synthesis.

The inhibiting effect of the nucleic acids according to the invention can be detected or used both in vitro and in situ, i.e., in living cells and also in an organism comprising these cells. The use can take place for diagnostic purposes and also for therapeutic purposes. This could be accomplished hitherto in this form according to the prior art by no other class of molecules such as antibodies, peptides, oligosaccharides or low-molecular substances, especially those which have been isolated from combinatorial libraries.

As already specified above, with the nucleic acids disclosed herein, inhibitors of guanine nucleotide exchange factors are provided, wherein it is notable that inhibitors for the small ARF-GEF's are provided for the first time by the nucleic acids according to the invention. These inhibitors thus typically or preferably comprise exogenous and/or comparatively small molecules which are different from those molecules or derivatives or mutants thereof with which the guanine nucleotide exchange factor interacts in situ. The latter generally comprise proteins and especially an ADP ribolysation factor or ADP ribosylation factors. Especially herein, inhibitors should not be understood as those which for example consist of the guanine nucleotide exchange factor itself, mutated variants of the guanine nucleotide exchange factor, parts of the guanine nucleotide exchange factor, in situ interacting proteins such as the ARF proteins, mutated variants of in situ interacting proteins or parts of in situ interacting proteins. Such protein constructs can be used as transdominant negative inhibitors like the isolated PH domain of cytohesin-1. The use of the isolated PH domain as a transdominant negative inhibitor of cytohesin-1 is described herein and in Nagel W. et al. (J. Biol. Chem. 9 (1998), 14853-14861). In these methods, for example, the inhibition is based on the complexing of a natural interactor of the guanine nucleotide exchange factors in situ or the complexing of the endogenous guanine nucleotide exchange factor itself. In this connection however, it is also within the scope of the present invention that the nucleic acid which acts as an inhibitor according to the invention as such or in derivatised form is already part of the transcriptome. Transcriptome is herein understood as the entirety of all the transcribed ribonucleic acids of a cell or an organism. In other words, it is within the scope of the present invention that the nucleic acid according to the invention and thus also the inhibitor according to the invention is identical to a nucleic acid sequence which is naturally present in a cell, which however does not necessarily bind to small ARF-GEF's which can have various causes. One such possible cause can be the fact that the sequence occurring in the cell is localised at another site or in another compartment of the cell to the ARF-GEF's. Another possible cause can be the different time of expression of the ARF-GEF's relative to that of the said nucleic acid. Yet another cause can consist in the fact that the quantity of nucleic acid is not sufficient to bind to the ARF-GEF's to an extent that a function of the ARF-GEF's is impaired by the binding event.

Without wishing to be restricted thereto in the following, the numerous medical uses of the nucleic acid according to the invention appear to be based on the following interaction. This mechanism is described with reference to cytohesin, especially cytohesin-1, but fundamentally applies to all the guanine nucleotide exchange factors described herein.

According to the current understanding, cytohesin-1 plays a critical role in controlling the adhesion of leukocytes and controlling the dynamic regulation of the cytoskeleton. Cytohesin-1, a cytoplasmic 47 kDa protein, is specifically formed in leukocytes of the immune system (Kolanus, W., et al., Cell 86 (1996), 233-242). This regulatory protein consists of an aminoterminal coiled-coil structure, a central Sec7 domain, and a carboxyterminal PH (“pleckstrin homology”) domain on which borders a polybasic C domain. Cytohesin-1 is a key regulator of the β2 integrins present on leukocytes. By interaction with the cytoplasmic domain of the β2-subunit, cytohesin-1 activates the adhesion of the extracellular domains of the integrin LFA-1 (αL/β2; CD11a/CD18) to its specific ligands ICAM-1 (“intercellular adhesion molecule 1”) (Kolanus and Zeitimann, Curr. Top. Microbiol. Immunol. 231 (1998), 33-49). The interaction between cytohesin-1 and the intracytoplasmic domains of the β-2 integrin could be detected by two hybrid experiments (Kolanus, et al., Cell 86 (1996), 233-242). In this connection cytohesin-1 is recruited to the cytoplasmic membrane by cooperative interaction of the polybasic C peptide and the PH domain with the membrane phospholipid phosphatidylinositol-3,4,5-trisphosphate which is formed after stimulation of the leukocytes e.g. by the T cell receptor from the enzyme phosphoinositide 3-kinase (Nagel, et al., J. Biol. Chem. 9 (1998), 14853-14861; Venkateswarlu, et al., J. Cell. Sci. 112 (1999), 1957-1965). There, the interaction of the Sec7 domain of cytohesin-1 with the β2-integrin subunit results in activation of the β2-integrin receptors (Kolanus, et al., Cell 86 (1996), 233-242). Overexpression of cytohesin-1 or subdomain constructs of the Sec7 domain in a Jurkat E6 cell line results in constitutive adhesion of the LFA-1-integrin (αL/β2) to ICAM-1. However, if the isolated PH domain of cytohesin-1 becomes overexpressed in leukocytes, the cells can no longer adhere to ICAM-1 after a stimulus. This dominant negative effect cannot be observed for a mutant of the PH domain (R281C), in which arginine at position 281 is exchanged for cysteine (Nagel, W. et al., J. Biol. Chem. 9 (1998), 14853-14861). This effect is explained by the blocking of the interaction of the endogenous cytohesin-1 with phosphatidylinositol-3,4,5-trisphosphate by the isolated overexpressed PH domains. The recruiting of the cytohesin-1 to the cytoplasmic membrane and the consequent activation of the LFA-1 integrin is thereby prevented. The nucleic acids according to the invention can inhibit the association with the cytoplasmic membrane in a similar fashion by the direct inhibition of the PH domain of the endogenous cytohesin-1.

A connection could be established with another function in cell culture studies of the GDP/GTP exchange function of the Sec7 domain of cytohesin-1. A mutant form of cytohesin-1 in which glutamate was exchanged for lysine by a point mutation at position 157 (mutant E157K). The mutant E157K can no longer catalyse the GDP/GTP exchange in vitro. It was found that the overexpression of E157K inhibits the adhesion of leukocytes on ICAM-1 coated surfaces and influences the morphological phenotype. Despite this it was found that both with the overexpression of cytohesin-1 and also E157K, an antibody epitope corresponding with the activation of the LFA-1 integrin is induced (Geiger et al., C., EMBO J. 16 (2000), 3525-2536). The antibody mAb24 which recognises the activation epitope was described by Stewart and colleagues (Stewart et al., J Immunol 156 (1996), 1870-1817). As described in the present invention in the examples, the nucleic acids according to the invention and the mutant E157K inhibit the restructuring of the cytoskeleton. According to the present state of the art, the Sec7 domain of cytohesin 1 intervenes in two separate steps of the adhesion of leukocytes which are both required for the firm attachment of the cells to the surface: i) activation of the β-2 integrins and ii) subsequent restructuring of the cytoskeleton. The nucleic acids according to the invention block both mechanisms, the second mechanism, the mechanism dependent on the ARF-GEF activity, being further described in the examples.

As a result of the specific inhibition of the ARF-GEF activity of cytohesin-1 and the inhibition of the adhesion of leukocytes to surfaces, there are numerous possible applications of the nucleic acids according to the invention, especially in the medical field, which are ultimately based on the blocking of this interaction by the nucleic acids according to the invention. Furthermore, it also follows from the specificity described previously that the various applications are also transferable to those guanine nucleotide exchange factors which also have a Sec7 domain or a domain homologous thereto and thus to the group of small GEF's having a molecular weight of around 50 kDa or the GEF's not inhibitable by Brefeldin A to which, among others, in addition to cytohesin-1, cytohesin-2, cytohesin-3 and cytohesin-4 also belong.

Cytohesin-2 (ARNO) can catalyse in vitro the GDP/GTP exchange of ARF-1 and ARF-6 localised at the cytoplasmic membrane and influences the structuring of the actin cytoskeleton (Frank, S. R. et al., Mol. Biol. Cell 9 (1998), 3133-3146; Venkateswarlu K. et al., Curr Biol 8 (1998), 463-466). Cytohesin-3 (GRP1/ARNO3) catalyses the GDP/GTP exchange of ARF-1 in vitro, ARF6 in vivo and is connected with controlling the organisation of the Golgi apparatus (Franco, M. et al., Proc. Natl. Acad. Sci USA 95 (1998), 9926-9931; Langille et al., J. Biol. Chem. 274 (1999), 27099-27104). It was furthermore found that cytohesin-3 is selectively induced in anergic T-helper cells (Th1) (Korthauer et al., J. Immunol. 164 (2000), 308-318). The newest member of the ARF-GEF family, cytohesin-4, is mainly found in leukocytes but not in most other tissue (Ogasawara et al., J. Biol. Chem. 275 (2000), 3221-3230). The nucleic acids according to the invention can also be used in connection with these cytohesins, the use consisting in inhibition of the function, especially the afore-mentioned function(s) of the respective cytohesins.

The possible applications of the nucleic acids according to the invention, especially in the field of medicine, are also obtained from the type of participation of the β-2-integrin-mediated adhesion in the various clinical pictures or pathogenicity mechanisms. As already stated above, the β-2-integrin-mediated adhesion of the leukocytes can be prevented by the blocking of the binding properties of the ARF-GEF's, especially their Sec7 domains by means of the nucleic acids according to the invention. Consequently, the nucleic acids according to the invention can be used for the treatment of diseases based on the adhesion of leukocytes as described in the following. In addition, by using the nucleic acids according to the invention a screening system can be constructed in which screening is carried for compounds, especially small molecules, which also have an inhibitory effect comparable to that of the nucleic acids according to the invention.

The β-2 integrins occurring exclusively on leukocytes have an essential function in the regulated adhesion of immune cells. These processes control, among others, the cytotoxicity of T lymphocytes and NK (“natural killer”) cells, the migration of leukocytes to inflamed tissue, phagocytosis and the chemotactic behaviour of myeloid cells. A characteristic feature of integrins is the regularability of the binding to their specific ligands. The heterodimer integrins consist of an α subunit and a β subunit. Each β chain is capable of pairing with various α-subunits and thus forming a plurality of receptors with individual specificities. Depending on the β subunit found in the dimer, the integrins are divided into a plurality of subfamilies. So far 16 different α subunits and 8 different β subunits have been identified in vertebrates. Since the β-2-integrin-mediated adhesion of immune cells has central importance in the regulation of the immune system, the inhibition of this event is the starting point for a therapeutic intervention for clinical indications such as acute inflammations, reperfusion injury or organ transplantation (Schürmann, Chirurg 68 (1997), 477-487). Without being restricted to the examples described in the following, a reduction in tissue damage by reduction of β-2-integrin activity could be detected in reperfusion injury to the myocardium (Jones S. P. et al., Am J Physiol Heart Circ Physiol 279 (2000), H2196-2201; Lefer A. M., Ann Thorac Surg 68 (1999), 1920-1923), the kidneys (Tajra L. C. et al., J Surg Res 87 (1999), 32-38) or the cerebral blood vessels (Prestigiacomo C. J. et al., Stroke 30 (1999), 1110-1117). An extensive therapeutic potential is thus opened up for the inhibition of β-2-integrin-mediated adhesion of leukocytes in transplantation medicine or the treatment of myocardial infarcts or strokes.

The blocking of the β-2-integrin-mediated adhesion of leukocytes by means of the nucleic acids according to the invention can also be a therapeutic starting point for the treatment of acute or chronic diseases. As non-limiting examples, mention may be made of rheumatoid arthritis (Issekutz A. C., Inflamm Res 47 Suppl 3 (1998), 123-132; Torrsteinsdottir I. et al., Clin Exp Immunol 115 (1999), 554-560) or inflammatory diseases of the skin such as psoriasis (Schon et al., J Invest Dermatol 114 (2000), 976-983). In these cases blockades of the leukocyte adhesivity could lead to a reduction of the inflammatory processes and an improvement in the clinical symptoms.

In the same way, integrins are ascribed a role in the metastasis of tumours, e.g. in the case of lymphomas, leukaemias or melanomas (Giancotti, F. G. and Mainiero, F., Biochim Biophys Acta 1198 (1994), 47-64). A decisive step in this process is the attachment of the transformed cells circulating in the bloodstream to the target tissue. This control can be mediated by adhesion molecules on the surfaces of the cells. For example, it can be shown that lymphoma cells with high LFA-1 expression show a greater tendency to metastasis than cells with a lower expression level. In this case, blocking of the adhesion could lower the metastatic potential of the lymphoma cells as has already been shown by the blocking of the extracellular β-2-integrin subunit with antibodies (Zahalka M. A. et al., J Immunol 150 (1993), 4466-4477). In the same way, a connection between modified β-2-integrin expression and the metastatic potential is also postulated for other types of cancers such as leukaemias (Puig-Kroger A. et al., J. Biol Chem 275 (2000), 28507-28512) or melanomas (Menter D. G., Immunol Cell Biol 73 (1995), 575-583). It is thus suggested that by inhibiting the attachment of the disseminating cells, the metastatic potential could be lowered for certain types of tumours.

A further aspect of the invention relates to a method, the so-called in vitro selection, with the aid of which specific nucleic acid ligands or aptamers can be isolated from a combinatorial library of various nucleic acid sequences for Brefeldin-A-insensitive small ARF-GEF proteins. Furthermore, the invention also comprises the uses of the nucleic acid ligands according to the invention described herein, especially the inhibition of the ARF-GEF activity in vitro or in cells, the use of the nucleic acid ligands for the detection of small ARF-GEF proteins, the use of the nucleic acid ligands for the isolation of their target proteins, the use of the nucleic acid ligands for the purposes of therapy wherein the application of the nucleic acid ligands can be achieved by exogenous addition or by expression of the nucleic acid ligands of suitable vectors in the cells.

As will be explained in greater detail in the examples, the nucleic acids according to the invention were obtained by means of so-called in vitro selection starting from a combinatorial library of different nucleic acid sequences. The nucleic acids obtained by the in vitro selection which bind to a target molecule are generally and also herein designated as nucleic acid ligands. The techniques for isolation of nucleic acids by in vitro selection are known to the person skilled in the art and are described in the literature (Methods Enzymol. 267 (1996); Klug and Famulok, Mol. Biol. Rep. 20 (1994), 97-107; Conrad et al., Mol Div. 1 (1995), 69-78; Williams and Bartel, Nucleic Acid Mol. Biol. 10 (1996), Soukup and Breaker, Curr. Opin. Struct. Biol. 10 (2000), 318-325). The method for in vitro selection basically thus involves the procedure that a plurality of different nucleic acid sequences (DNA, RNA and others) are brought in contact with a target and the nucleic acid(s) binding to the target is/are isolated, the non-binding nucleic acids preferably being removed, and then this/these nucleic acid(s) which may be modified in their sequence and thus a plurality of nucleic acids being again made available, is again brought in contact with the target. These steps can be repeated many times.

Nucleic acids or nucleic acid ligands in the sense of this invention preferably comprise DNA, RNA or chemically modified forms of DNA or RNA. More preferably the nucleic acid ligands belong to one of the classes of aptamers, aptazymes, ribozymes, intramers, natural nucleic acid ligands or are naturally occurring protein-binding nucleic acids. Especially preferred in this connection are the aptazymes, intramers and aptamers, of which three the group of intramers is in turn especially preferred. Natural nucleic acids should herein also be understood as those nucleic acids, whether as starting materials (partly or completely) for in vitro selection methods or whether as nucleic acids according to the invention, which have a sequence contained in the transcriptome of a cell or an organism, wherein transcriptome is herein understood especially as the entirety of the expressed nucleic acids. If natural nucleic acids are used, it is especially preferred if, instead of randomised libraries, natural libraries are used in selection processes, such as cDNA fragments of transcribed RNAs.

In vitro selection should herein especially be understood as the process by which the nucleic acid ligands according to the invention, preferably from the group of aptamers, intramers, aptazymes or ribozymes, more preferably from the group of aptamers and aptazymes, can be isolated from combinatorial libraries of nucleic acids having different sequences by in vitro selection.

Aptamers (Ellington and Szostak, Nature 346 (1990), 818-822) in the sense of this invention are to be understood as single-stranded nucleic acid ligands isolated from combinatorial libraries of randomised nucleic acids by in vitro selection. This process is sometimes also called SELEX (“systematic evolution of ligands by exponential enrichment”) (Tuerk and Gold, Science 249 (1990), 505-519). These aptamers consisting of ssDNA or ssRNA possess very high affinities and specificities for their antigens. To date nucleic acid ligands have been isolated for small organic compounds, peptides, proteins or even complex structures such as viruses and cells (Review article: Gold et al., Annu. Rev. Biochem. 64 (1995), 763-797; Ellington and Conrad, Biotechnol. Annu. Rev. 1 (1995), 185-214; Famulok, Curr. Opin. Struct. Biol. 9 (1999), 324-329). The high potential of the technology lies in the aptamer selection process which takes place purely in vitro. The nucleic acid ligands are enriched from combinatorial libraries of up to 10¹⁵ individual sequences by repeated cycles comprising contact with the antigen, separation of all non-binding nucleic acids and enzymatic amplification of the molecules interacting with the target.

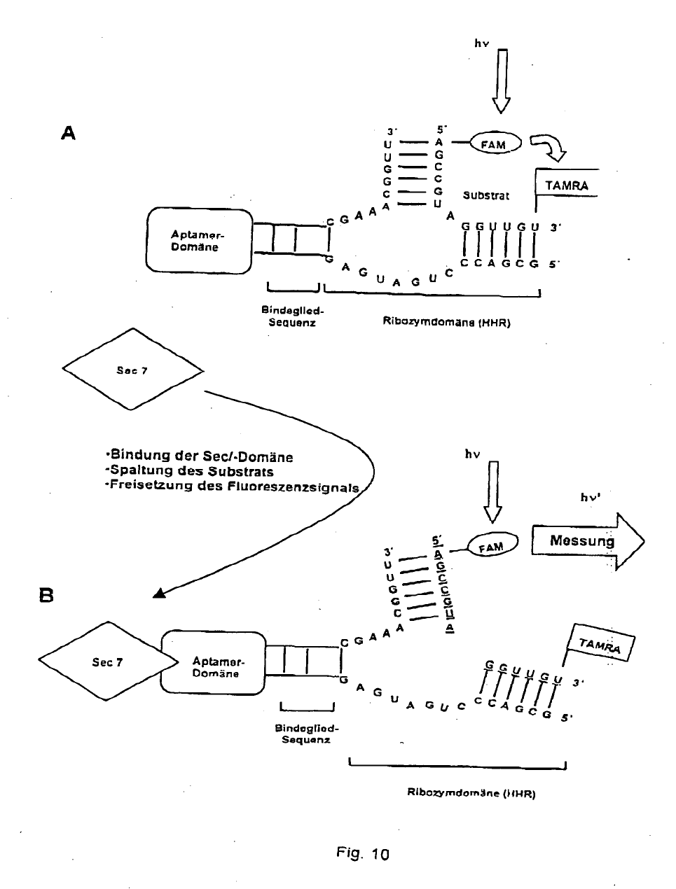

Aptazymes in the sense of the present invention are hybrid molecules from ribozymes and aptamers and accordingly consist of a catalytic and a ligand-binding nucleic acid domain. In this connection the activity of the ribozyme is regulated, i.e., activated or inhibited by the binding of the ligand to the ligand-binding nucleic acid domain (allosteric centre). The aptazyme technology is known to the expert and is described for example in Soukup and Breaker, Curr. Opin. Struct. Biol. 10 (2000), 318-325. In this connection aptazymes can be constructed modularly by rational design from a ribozyme part and the allosteric centre or isolated de novo by in vitro selection. For example, an aptazyme was constructed from a hammerhead ribozyme, a small catalytic motif which can cleave RNA sequences sequence-specifically (Forster and Symons, Cell 49 (1987), 211-220; Haseloff and Gerlach, Nature 334 (1988), 585-591), and an aptamer domain which binds specifically to ATP such that the binding of ATP resulted in an allosteric inhibition of the catalytic activity (Tang and Breaker, Chem. Biol. 4 (1997), 453-459, Tang and Breaker, Nucleic Acid Res. 26 (1999), 4222-4229). On the other hand, aptazymes could also be isolated by in vitro selection from combinatorial libraries of different nucleic acid sequences which contained a new type of binding domain for a ligand (Robertson and Ellington, Nat. Biotechnol. 17 (1999), 62-66, Koizumi et al., Nat. Struct. Biol. 6 (1999), 1062-1071). Aptazymes in the sense of this invention can for example be those which contain a ligand-binding site for small ARF-GEF proteins. Such aptazymes can be produced on the basis of the nucleic acids according to the invention by constructing these modularly with a ribozyme part and the allosteric centre. A schematic diagram of an aptazyme is shown in FIG. 10.

Natural nucleic acid ligands should also be understood herein in addition to the aforesaid as those nucleic acids, nucleic acid ligands or parts thereof, i.e., nucleic acid motifs, which as such are encoded in the genomes of organisms and possess the capability of binding to a target protein. Examples among many others are HIV-Tar-RNA which binds to the HIV Tat-protein or HIV-RRE-RNA, which binds to the HIV-REV protein. A wealth of such natural nucleic acid ligands are known to the person skilled in the art. Hitherto unknown nucleic acid ligands for certain target proteins can also be isolated by in vitro selection from combinatorial libraries of natural nucleic acids of different sequence which for example represent the genomic RNA of an organism, an organ or a cell. The manufacture of such combinatorial libraries is known to the person skilled in the art and is described, for example, in Singer et al., Nucleic Acid Res. 25 (1997), 781-786. In this way, for example, genomic DNA sequences which bind the transcription factor TFIIIA of Xenopus laevis can be isolated (Kinzler and Vogelstein, Nucleic Acid Res. 17 (1989), 3645-3653). In another experiment, ligands for the bacteriophage MS2 protein could be isolated from mRNA fragments from the transcriptome of the bacterium E. coli (Shtatland et al., Nucleic Acids Res. 28 (2000), E93). In this connection, surprisingly new, hitherto unknown interactions, for example, with the mRNAs of the rffG gene or the ebgR gene were found. This shows that new nucleic acid ligands for proteins can be isolated from libraries constructed from the transcriptome of an organism.

Intramers should herein be understood as those aptamers or natural nucleic acid sequences or natural nucleic acid ligands which in addition to the pure binding of their ligand have been specifically constructed for use in the intracellular milieu. Quite generally the procedure in this connection is that further sequences are added to the nucleic acids according to the invention which have the result that the nucleic acid according to the invention is stable and/or expressed in an intracellular milieu. Typically, such additional nucleic acids which satisfy the aforementioned requirements or impart the corresponding required properties can already be added as a result of inserting the nucleic acid according to the invention in an expression system. For example, by using purely viral expression systems aptamers can be stabilised by attachment of viral RNA sequences and using viral promoters of a combined system of the vaccinia virus and the T7 bacteriophage, transcribed with a high transcription efficiency and localised in the cytoplasm by viral polymerases and bind their target molecules there (Blind et al., Proc. Natl. Acad. Sci. USA 96 (1999), 3606-3610). Intramers should herein especially be understood as those in vitro selected aptamers which are optimised and used by stabilisation, localisation, expression, attachment of natural and non-natural nucleic acid sequences, chemical modifications or other measures for the purpose of functional modulation of the activity of an intracellular target molecule. Other expression systems which can be used for the transcription of intramers are, for example, eukaryotic RNA-polymerase-II-dependent systems. Thus, aptamers were inducibly expressed in transgenic flies against a protein (B52) involved in splicing in Drosophila melanogaster via a heat shock promoter (Shi H. et al., Proc. Natl. Acad. Sci USA 96 (1999), 10033-10038). In the same way, many expression systems are known which were used for the transcription of intracellular ribozymes. For example, RNA-polymerase-III promoters were used for the transcription of the RNA ribozyme. For this purpose the functional ribozyme sequences were mainly used in the context of parts of small natural RNA molecules in cis. By attachment of compact sequences of tRNA or U6-RNA very high transcription yields of ribozyme transcripts could be achieved (Lee et al., Gene Ther. 2 (1995),377-384, Zouh et al., Thompson et al., Nucleic acid res. 23, 2259-2268; Bertrand et al., RNA 3 (1997), 75-88). Further polymerase-III promoters suitable for the expression of RNA in cells are known to the person skilled in the art and are described in the literature (review article: Couture and Stinchcomb, TIG 12 (1996), 510-514; Rossi, Tibtech 13 (1995), 301-305; Bramlage et al., Tibtech 16 (1998), 434-438). They are responsible for the transcription of many small RNA molecules in eukaryotic cells and are active in all cell types. In addition, they can be found ubiquitously in homologous RNA transcription units in all eukaryotic species. Pol-III promoters control the transcription of small RNA such as tRNA, snRNA, snoRNA, 5S rRNA, or small viral RNA, such as the adenoviral VA-RNA. Another advantage of the RNA-Pol III promoters is their extremely high transcription activities. Natural transcripts accumulate in cells up to 10⁵ to 10⁶ molecules per cell. Examples of RNA-polymerase III promoters are the promoters of 5S-RNA genes (type 1) (Specht et al., Nucleic Acid Res. 19 (1991), 2189-2191), tRNA promoters (type 2) (Thompson et al., Nucleic Acid Res. 23 (1995), 2259-2268; Sullenger et al., J. Virol. 65 (1991), 6811-6816) U6 snRNA (Type 3) (Das et al., EMBO J. 7 (1988), 503-512, Lobo and Hernandez, Genes Dev. 58 (1989), 55-67) or other Pol-III promoters not coming under the classification 1 to 3 such as the 7SL-RNA promoters for example (Bredow et al., Gene 86 (1989), 217-225). Some of these promoters, among others tRNA, U6 snRNA or VA1-RNA promoters, have conventionally been used for the expression of RNA molecules (Review article: Rossi, Tibtech 13 (1997), 301-305; Bramlage et al., Tibtech 16 (1998), 434-438).

Another suitable expression system is the so-called Pol III expression system. The Pol III expression system comprises in this connection a nucleic acid sequence comprising the following elements in the 5′-3′ direction:

a C1 motif,

an A1 box,

a C2 motif,

an A3 box, and

a terminator,

wherein the C1 motif and the C2 motif together form a helix;

the A1 box comprises k bases wherein k is independent of l and m is an integer between 0 and 100;

the A2 box comprises l bases wherein l is independent of k and m is an integer between 0 and 100, and

the A3 box comprises m bases wherein m is independent of k and l is an integer between 0 and 20.

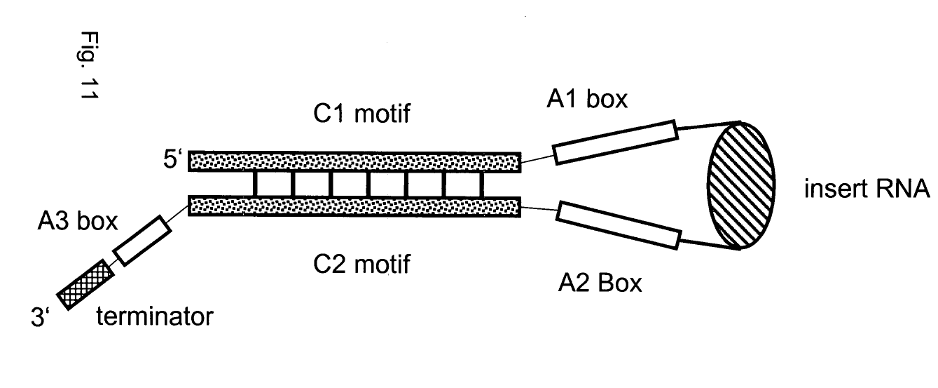

Such a Pol III expression system is shown in FIG. 11.

In order that the possibilities of the aptamer technology can be used more intensively, chemical derivatives of the deoxyribonucleic and ribonucleic acids can be developed. For example, the nucleic acids can be stabilised against degradation by nucleases. Such stabilisation can be achieved at various sites of the nucleic acid molecules. Thus, it is possible to modify the connecting group between the nucleobases, i.e., the phosphodiester bond, but also the sugar residue or the bases or combinations of these measures. The various possibilities for producing chemically modified nucleic acids are known to the person skilled in the art and are described in detail in the literature (Review article: Uhlmann E. and Peyman A., Chem Reviews 90 (1990), 543-584; Eaton B. E. and Pieken W. A., Annu Rev Biochem 64 (1995), 837-863; Eaton B. E., Curr Opin Chem Biol 1 (1997), 10-16). Especially preferred are the 2′-amino- and 2′-fluoro-deoxyribonucleotides compatible with the in vitro transcription reaction of the selection process. In the presence of nucleases, the half-lives of the modified molecules compared with natural nucleic acids increase from minutes to a few hours (Lee S.-W. and Sullenger B. A., Nat Biotechnol 15 (1997), 41-45; Kubik et al., J. Immunol. 159 (1997), 259-267; Pagratis et al., Nat. Biotechnol. 15 (1997), 68-73). It has also been shown that the permanent modification of the sugar residues of purines with 2′-O-methyl groups and the attachment of short phosphorothioate groups to the 5′- and 3′-ends can significantly increase the stability of RNA aptamers in biological media (Green et al., Chem. Biol. 2 (1995), 683-695). The ribonucleic acid ligands attain half-lives of up to 72 hours.

Nucleic acid ligands are structurally stabilised by introducing chemical connecting groups. An example is the structural stabilisation of two short oligonucleotides from the minimal RBE (Rev binding element) by a connecting stilbene dicarboxyamide bridge (Nelson et al, Biochemistry 35 (1996), 5339-5344). The RBE in the genomic RNA of the HIV virus binds to the viral Rev protein. Rev is thereupon responsible for the transport of the viral RNA from the cell nucleus into the cytoplasm. Without the chemical link, the two oligonucleotides were not capable of constructing thermally stable structures via base pairing. With the stilbene dicarboxyamide bridge the structure was stabilised, and bound the Rev protein with the same affinities as the viral RBE. This method is especially useful for producing severely shortened functional nucleic acid ligands.

The nucleic acids according to the invention are fundamentally, and also in the various possible uses or applications and especially those disclosed herein, not restricted to the specific disclosed sequences. Rather it is within the scope of the capabilities of the persons skilled in the art, starting from the specifically disclosed nucleic acids, to modify these. Such nucleic acids are herein also designated as modified nucleic acids. Whenever the nucleic acids according to the invention and their application or use are mentioned herein or in the claims, this at the same time always includes the modified nucleic acids and their application or use. Within the scope of such modification, nucleobases can be chemically modified or individual or a plurality of the nucleobases can be exchanged or deleted. The production of nucleic acids in which nucleobases are exchanged or deleted and which despite this exhibit the same binding properties as the original nucleic acid ligands is known to the person skilled in the art and is described in the literature. An example is the production of mutated and shortened derivatives of an aptamer against the transcription factor NFκB (Lebruska and Maher, Biochemistry 38 (1999), 3168-3174). In order to isolate mutants of an aptamer (aptamer 3) which bind to the transcription factor NFκB like the original aptamer 3, a library of mutants was produced which contains the correct base at each position with a probability of 0.7 and the other three bases with a probability of 0.1 each. Mutants were isolated by in vitro selection which, despite exchange of nucleobases, still bound to the transcription factor. In order to produce deletion, the original aptamer was partially cleaved by alkali hydrolysis and in binding studies those fragments were isolated which despite the deletions still bound to the transcription factor NFκB. In this way shortened forms of the original aptamer could be produced. However, further nucleotides can also be added to the sequences according to the invention, as already stated in connection with the aptazymes and intramers. In this respect, the specific sequences of the nucleic acids disclosed herein represent a framework starting from which further sequences are feasible and which is only limited by the fact that the nucleic acid modified to such an extent binds to the guanine nucleotide exchange factor as before and in preferred embodiments also modulates, especially inhibits, the GDP/GTP exchange function.

The guanine nucleotide exchange factor can fundamentally, and also in the various possible uses or applications and especially those disclosed herein, comprise such a one that belongs to the group of guanine nucleotide exchange factors for ADP ribosylation factors (ARF) having a molecular weight of around 50 kDa or less. The ADP ribosylation factors belong to a family of regulatory GTPases which are also designated as G proteins. The guanine nucleotide exchange factor can also comprise such a one that is not inhibitable by Brefeldin A. Furthermore, the guanine nucleotide exchange factor can comprise such a one selected from the group comprising cytohesin-1, cytohesin-2 (ARNO), cytohesin-3 (GRP-1, ARNO3), cytohesin-4 or EFA6, wherein cytohesin-1 and cytohesin-2 are quite especially preferred. Finally, the guanine nucleotide exchange factor can also be such a one which has a Sec7 domain or a homologue thereto. Finally, the guanine nucleotide exchange factor can comprise such a one which comprises an arbitrary combination of two or a plurality of the afore-mentioned features.

In another aspect the invention relates to an expression system comprising at least one or a plurality of nucleic acids according to the invention or modifications thereof. Expression system should herein be especially understood as vectors which contain suitable expression cassettes for the nucleic acids according to the invention and which make possible the infiltration and/or the expression in cells of the nucleic acids according to the invention. Especially preferred here are eukaryotic cells. Suitable vectors for transfection of genes are known to the person skilled in the art. These can comprise mere DNA vectors such as plasmids, cosmids, BACs (“bacterial artificial chromosomes”), YACs (“yeast artificial chromosomes”), MACs (“mammalian artificial chromosomes”). Furthermore, viral vectors such as Sindbis viruses, vaccinia viruses, adenoviruses, AAV or retroviruses are also possible. Furthermore, the invention relates to expression cassettes which comprise at least one of the nucleic acids according to the invention or the genes for the nucleic acid ligands according to the invention, preferably RNA ligands, as described in Example 4 (see also Blind et al., Proc Natl. Acad. Sci. USA 96 (2000), 3606-3610; PCT/EP/00/02727). Other examples of expression cassettes are described in Good et al., Gene Ther. 4 (1997), 45-54 or Bertrand et al., RNA 3 (1997), 75-88. In this connection such expression cassettes typically comprise suitable promoters for the expression of nucleic acid ligands and in addition to the nucleic acid ligands can additionally code for sequences which impart stability to the hybrid molecule comprising nucleic acid ligand and additional sequences or control its intracellular localisation (Blind and Famulok, DE 100 46 913.2).

The nucleic acids according to the invention can be used as such and also in expression systems or expression cassettes. A particularly important application is the area of medicine wherein both therapeutic and diagnostic applications are possible, based on the interactions and mechanisms described herein. In this respect the nucleic acids according to the invention can be used singly or in arbitrary combinations for the manufacture of a medicament. Quite especially preferred is the use for manufacturing a medicament for the prevention and/or treatment of diseases or clinical pictures wherein the medicament influences the β-2 integrin-mediated adhesion of immune cells. The immune cells preferably comprise those cells selected from the group comprising leukocytes, phagocytes, T-lymphocytes, myeloid cells and NK (“natural killer”) cells.

The use of the nucleic acids according to the invention can take place in therapy both in the direct administration of the same and also as part of gene therapy. In the case of gene therapy, the vectors, expression systems and expression cassettes containing the nucleic acids according to the invention are of particular importance. In the gene-therapeutic use of the nucleic acids according to the invention, the fundamental application aspect in this connection is that an expression vector containing the nucleic acids according to the invention is transfected by a suitable vector in cells of a eukaryotic organism, especially in human cells. From the transfected expression cassette the nucleic acids according to the invention can then be expressed in the cells and there inhibit the function of the ARF-GEF proteins. As vectors for gene-therapeutic attempts it is possible to use free DNA, DNA packed in carrier materials such as cationic liposomes, which are described in greater detail below, or viral vectors such as adenoviruses, adeno-associated viruses, retroviruses, herpes viruses, polyomaviruses, papillomaviruses or vaccinia viruses (review article: Walther and Stein, Drugs 60 (2000), 249-271; Buchschacher and Wong-Staal, Blood 95 (2000), 2499-2504; Krauzewicz and Griffin, Adv. Exp. Med. Biol. 465 (2000), 73-82; Virtanen et al., Adv. Exp. Med. Biol. 465 (2000), 423-429). Suitable vectors for the nucleic acid sequence according to the invention are known to the persons skilled in the art. As free DNA vectors among others it is possible to use plasmids and cosmids which are infiltrated into the cells by electroporation, lipofection or CaPO₄ precipitation (review article: Gregoriadis, Pharm. Res. 15 1998, 661-670). An extension of this method is the use of episomal replicating plasmids which carry the “origin of replication” of the Epstein-Barr virus (OriP) and express the EBNA-1 antigen. These vectors replicate extrachromosomally in primate and dog cell lines and can persist permanently there (Yates et al., Nature 313 (1985), 812-815; Chittenden et al., J. Virol. 63 (1989), 3016-3025). Another method of replicating foreign genetic material permanently in eukaryotic cells is the use of so-called minichromosomes. These large DNA molecules carry centromer and telomer sequences as natural chromosomes and are duplicated in mitosis and transmitted to the daughter cells. Their size (in the megabase range) additionally allows the cloning of very large DNA fragments of several 100 000 bases. Minichromosomes have been developed for bacteria, yeast and mammalian cells (YACs and BACs, see: Grimes and Cooke, Hum. Mol. Genet. 7 (1998), 1635-1640; Amemiya et al., Methods Cell. Biol. 60 (1999),:235-258; Brown et al., Trends Biotechnol. 18 (2000), 218-223). The vectors specified here are not limiting examples for vector systems which are described in the literature and known to those skilled in the art.

Frequently used viral vectors are retroviruses whose RNA genome after reverse transcription integrates DNA stably into the genome of the host. Most frequently used are MoMuLV-based vectors which however can only infect proliferating cells. Thus, systems based on lentiviruses (among others HIV) have been developed with which non-dividing cells can also be infected (Review article: Miller, Hum Gene Ther. 1 (1990), 5-14; Gordon and Anderson, Curr. Opin. Biotechnol. 5 (1994), 611-616). Another frequently used class are the adeno-associated viruses (AAV). These ssDNA viruses are distinguished among other things by the advantage that they can integrate genetic material at a defined site in chromosome 19 (review article: Grimm and Kleinschmidt, Hum. Gene. Ther. 10 (1999), 2445-2450).

The direct administration of the nucleic acids according to the invention, but also if necessary the administration as part of gene therapy, can take place in this connection by means of any galenic techniques and compositions which comprise one or a plurality of the nucleic acids according to the invention in their various embodiments. As examples, reference may be made here to solutions, powders, tablets, gels and the like. In this case the nucleic acid according to the invention is typically manufactured and/or administered together with a stabiliser, adjuvant, pharmaceutically acceptable carrier, preservative, carrier or a combination thereof. Methods for infiltrating the nucleic acids according to the invention into cells are known to the person skilled in the art. The nucleic acids according to the invention can be used directly for example as liposomal formulations. Transfection protocols with cationic liposomes are described in Ausubel et al. (Current Protocols in Molecular Biology (1991), Wiley Interscience, New York) or Sambrook et al. (Molecular cloning—a laboratory manual, 2^{nd} Ed. (1989), Cold Spring Harbor Laboratory Press, Cold Spring Harbor). Detailed descriptions of the type of cationic amphiphiles (liposomes) suitable for the in vivo administration of therapeutic nucleic acids are described in Felgner et al. (Proc Natl Acad Sci USA (1987), 7413-7417), Behr et al., Proc. Natl. Acad Sci USA 86 (1994), Epand et al., (U.S. Pat. No. 5,283,185) or Lee et al. (U.S. Pat. No. 5,925,628). Other transfection reagents for nucleic acids are for example dendrimers (Tang et al., Bioconjugate Chem 7 (1996), 703) or other polycationic polymers (e.g. described in Illum, U.S. Pat. No. 5,744,166). Another possibility for introducing nucleic acids into cells are hybrid molecules comprising the nucleic acid and short signal peptides which occur naturally, e.g., in the basic domain of the HIV Tat protein or helix 3 of the Antennapedia protein (Lit site). These signal peptides can infiltrate macromolecules directly through the cytoplasmic membrane into the intracellular compartment by means of a process hitherto not yet understood in detail. The death of such signal peptides was described for example by Troy and Shelanski (U.S. Pat. No. 5,929,042) or by Lin. and Hawiger. (U.S. Pat. No. 5,807,746). The cited reagents are used to describe the numerous possibilities for infiltration of nucleic acids in cells which are known to the person skilled in the art and should not be understood as limiting examples here.

With the nucleic acids according to the invention, an inhibitor for the guanine nucleotide exchange factors of the ARF proteins is provided for the first time, as they are described herein. The uses of this inhibitor correspond to those of the nucleic acids according to the invention. The same applies to the pharmaceutical development of a pharmaceutical composition or means especially containing the inhibitor which, in addition to the inhibitor, can also contain other pharmaceutically effective compounds. Furthermore, such a means can also contain conventional galenic adjuvants which are selected from the group comprising buffers, preservatives, chelators, stabilisers, masking agents, fillers, disintegrating agents, pharmaceutically acceptable carriers and isotonicity-adjusting compounds. Moreover, with the inhibitor according to the invention a tool is also available for generally representing and studying the intracellular events in connection with the interaction of ARF-GEF's, wherein as a result of the size of the inhibitor and its chemical or molecular nature compared with the use of modified interaction partners such as mutated or overexpressed interaction partners, interactions originating from said inhibitor on other systems inside the cell can be eliminated.

Another application of the nucleic acids according to the invention lies in the diagnostic area. Nucleic acid ligands like antibodies can be used in numerous diagnostic applications (Review article: Hesselberth et al., J Biotechnol 74 (2000), 15-25; Brody and Gold, Biotechnol 74 (2000), 5-13; Jayasena Clin Chem 45 (1999), 1628-1650; Osborne et al., Curr Opin Chem Biol 1 (1997), 5-9). As a result of the binding of the nucleic acids according to the invention to the target structure, i.e. the guanine nucleotide exchange factor, said factor can be labelled. If the nucleic acid according to the invention has a label for its part, this and thus the complex of guanine nucleotide exchange factor and nucleic acid according to the invention can be detected. Suitable labels are known to the person skilled in the art and comprise, among others, those selected from the group comprising radioactive labelling, labelling with fluorescent dye s, labelling with groups such as biotin or digoxigenin or labelling with enzymes such as β-galactosidase. Suitable fluorescent dyes for the labelling of nucleic acids are known to the person skilled in the art and comprise among others AMCA-X, fluorescein, rhodamine, Cy3, Cy 5, Cy 5.5, HEX, D-AMCA, tetramethylrhodamine or Texas Red. The detection of said complex with other nucleic/acid/protein complexes can be carried out for example by FACS (Ringquist and Parma, Anal Chem. 70 (1998), 3419-3425), fiberoptic microarray sensors (Lee and Walt, Anal Biochem 282 (2000), 142-146), capillary electrophoresis (German I., Anal Chem 70 (1998), 4540-4545) or fluorescence microscopy.

Alternatively the nucleic acids according to the invention, the guanine nucleotide exchange factor or both can be labelled with fluorescent dye s and the interaction between the nucleic acid and the guanine nucleotide exchange factor results in a measurable change in the fluorescence signal. For example, the binding of thrombin to anti-thrombin aptamers labelled with fluorescent dye s could be detected by measuring the change in the fluorescence anisotropy (Potyrailo R. A. et al., Anal Chem 70 (1998), 3419-25). In this case up to 0.7 amol of thrombin could be detected. In another embodiment one interaction partner, the nucleic acid or the guanine nucleotide exchange factor is labelled with a fluorescence-quenching group (quencher) and the other is labelled with a fluorophor group (donor) wherein the interaction between the two interaction partners results in quenching of the fluorescence emission of the donor by the quencher. If the interaction takes place, this can be measured by a reduction in the fluorescence signal. This method also known as FRET (“fluorescence resonance energy transfer”) is known to the person skilled in the art and is used for the detection of protein/protein interactions (Sorkin A. et al., Curr Biol. 10 (2000), 1395-1388; Latif R. and Graves P., Thyroid. 10 (2000), 407-412, 14: Buranda T. et al., Cytometry 37 (1999), 21-31), The principles of detecting interactions between molecules by fluorescence labelling and the various possible arrangements for detection using single or multiple fluorescence labelling of one or both interaction partners are familiar to the person skilled in the art.

In the same way, complexes of the nucleic acids according to the invention and the guanine nucleotide exchange factor can be detected on suitable sensors which are coated with one of the two binding partners. In this case, the detection can take place via the increase in mass after the binding of the second binding partner. For example, complexes of RNA aptamers and the enzyme 2′-5′-oligoadenylate cyclase or RNA aptamers and the NS3 protease of the hepatitis C virus have been detected by so-called SRP (“Surface Plasmon Resonance”) analyses (Hartmann et al., J Biol Chem 273 (1998), 3236-3246; Hwang et al., Biochem Biophys Res Commun 279 (2000), 557-562). Other suitable methods such as quartz crystal microbalance (QCM) (Kosslinger et al., Biosens Bioelectron 7 (1992), 397-404; Hengerer et al., Biosens Bioelectron 14 (1999), 139-144) are known to the person skilled in the art.

In a particular embodiment the interaction between the protein and the nucleic acid ligands, especially an aptazyme can be detected by a catalytic event. In this case, the nucleic acid domain recognising the ARF-GEF protein is linked to a ribozyme domain in such a way that the binding of the ARF-GEF protein allosterically influences, i.e., activates or inhibits, the activity of the ribozyme domain. The construction of signal-emitting aptazymes is known to the person skilled in the art and described in the literature (Hesselberth et al., J Biotechnol 74 (2000), 15-25; Marshal and Ellington, Nat Struct Biol 6 (1999), 992-994; Soukup and Breaker, Trends Biotechnol 17 (1999), 469-476). Examples of ribozyme domains which have been used in aptazyme constructs are effector-activated ribozyme ligases (Robertson and Ellington Nucleic Acids Res 28 (2000),:1751-1759) or hammerhead ribozymes (Soukup et al., J Mol Biol 298 (2000), 623-632). In the case of ribozyme ligases which themselves link to an oligonucleotide the active ribozyme domain can be detected by via reverse transcription PCR with a primer complementary to the linked oligonucleotide (Robertson and Ellington, Nat Biotechnol 17 (1999), 62-66). Active hammerhead domains can be detected by cleaving of a substrate oligonucleotide for example in a FRET assay (Jenne et al., Angew. Chem. 111 (1999), 1383-1386). In this case, the oligonucleotide to be cleaved is labelled with a fluorescence-emitting (donor) and a fluorescence-quenching group (quencher). In the uncleaved state donor and quencher are located in the immediate neighbourhood and the light emitted by the donor is absorbed by the quencher. After the cleaving the donor is released and the fluorescence now freely emitted by it can be detected.

In the same way the nucleic acid ligands according to the invention like antibodies can be used in ELISA applications known to the person skilled in the art. An example for the application of an aptamer in an Elisa application is the specific detection of the protein VEGF by means of anti-VEGF aptamers (Drolet et al., Nat Biotechnol 14 (1996), 1021-1024; Kato et al., Analyst 125 (2000), 1371-1373). The nucleic acids according to the invention can also be used for the detection of the ARF-GEF proteins on chips as carrier materials. The application of nucleic acid ligands in such formats was described for example in Brody et al. (Mol Diagn 4 (1999), 381-388). Suitable as sample materials among others are body fluids such as blood, urine, lymph fluid, vaginal fluid, cerebrospinal fluid, aspirate, stool, biopsies, cellular samples and suspensions thereof. This results in the specific detection of ARF-GEF proteins in the samples to be studied.

The nucleic acids according to the invention can also be used for these purposes by means of so-called kits. In the present case, such a kit comprises at least one of the nucleic acids according to the invention which are optionally already provided with one of the labels described above. Other embodiments comprise other reagents such as positive and negative controls, for example. Negative controls can be constructed so that the kit contains a nucleic acid which does not bind to guanine nucleotide exchange factors or certain parts thereof as also described herein or does not modulate, especially does not inhibit its function, especially the GDP/GTP exchange function. As a positive control in another embodiment it is possible to add a guanine nucleotide exchange factor to which the nucleic acid according to the invention, also present in the kit, binds. In one embodiment of the kit both the nucleic acid according to the invention and the guanine nucleotide exchange factor can be present either singly or jointly in a cell, preferably expressed or in an expressible form. In other embodiments the kit can furthermore comprise buffer, washing solutions and the like.

The use of the nucleic acids according to the invention in order to form a complex with the guanine nucleotide exchange factors for ARF proteins described herein can, in addition to the therapeutic and diagnostic area, also take place in the technical or preparative area. In this case, the nucleic acids according to the invention can be coupled to support materials such as sepharoses, agaroses or magnetic particles. These affinity support materials can then be used for preparative purification of the ARF-GEF proteins. Corresponding protocols such as affinity precipitations, similar to immune precipitations, or affinity chromatographies are known to the person skilled in the art. Protocols for analogous immune precipitations are described for example in Ausubel et al. (Current Protocols in Molecular Biology (1991), Wiley Interscience, New York) or Sambrook et al. (Molecular cloning—a laboratory manual, 2nd Ed. (1989), Cold Spring Harbor Laboratory Press, Cold Spring Harbor). After applying the sample and binding the interaction partner, the material can be freed from non-specific binding partners using suitable washing solutions. The specifically bound interaction partner, i.e., the guanine nucleotide exchange factor is then eluted and optionally quantified. In this connection, typically high salt concentrations, detergents such as SDS, chaotropic agents such as urea or guanadinium hydrochloride are used as eluents or elution conditions. Furthermore, the protein can be eluted by heat-denaturing of the nucleic acid ligands, typically between 60° C. and 95° C. or denaturing the structure of the nucleic acid ligands by complexing divalent ions, for example, by EDTA. Protocols which can be used for resolving the interaction between the nucleic acid ligands and the target protein to be purified are known to the person skilled in the art. The afore-mentioned samples can be used as samples. In addition, as a result of the preparative character of this embodiment, the sample can also be a culture medium for cells which form the respective binding partner, with or without cells (in the case where the binding partner is exported) or a suspension of cells. A protocol for the purification of human L-selectin expressed in CHO cells by means of a specific aptamer using affinity chromatography was described by Romig and colleagues (Romig, J Chromatogr B Biomed Sci Appl 731 (1999), 275-284).

Another form of application of the complex formation between one of the nucleic acids according to the invention and a guanine nucleotide exchange factor takes place in the screening method according to the invention which is described in the following.

In one aspect the invention also comprises the use of the nucleic acids according to the invention in screening systems. For example, libraries of substances, preferably low-molecular substances can be screened for those substances which block the interaction of the two interaction partners by binding to one of the two interaction partners, i.e., Brefeldin A-resistant ARF-GEF proteins or at least one of the nucleic acids according to the invention. Analogous screening systems were developed for example for the screening of protein/protein interactions. Thus, low-molecular inhibitors could be identified for the interaction between cyanovirin-N and the HIV protein gp120 (McMahon et al., J. Biomol. Screen 5 (2000), 169-176) or low-molecular inhibitors for the interaction between peptides of a phage library of the tyrosyl-tRNA synthase of H. influenza (Hyde-DeRuyscher et al., Chem. Biol. 7 (2000), 17-25). Molecules, i.e. more accurately inhibitors which displace the nucleic acid sequences according to the invention from the small ARF-GEF proteins could be functionally equivalent to the nucleic acids according to the invention. In addition to the use of the nucleic acids according to the invention as inhibitors, these can thus also be used for the identification of compounds from a library or for allocating a certain function or use to members of this library, namely those that have an effect functionally analogous to the nucleic acids according to the invention and modulate, more accurately, inhibit the interaction between guanine nucleotide exchange factor and ADP ribolysation factor. In addition to this use, the nucleic acids according to the invention in the same way as the members of the library identified in the screening method according to the invention, can also be valuable lead structures and can thus be used for the development of new types of therapeutic substances. In these screening systems the interaction between the nucleic acids according to the invention and the ARF-GEF proteins can also be detected, as already described, using fluorescence-based methods (e.g. FRET) or aptazyme constructs. During the displacement of the nucleic acids according to the invention by the substance present in the library a signal is generated. The substance thus identified binds to the same interaction centre as nucleic acids according to the invention, here the catalytic domain of the ARF-GEF protein. The identified substance is thus a functional analogue of the nucleic acid according to the invention with reference to the binding and with increased probability is a substance which can also inhibit the activity of the ARF-GEF protein by binding to the active centre. An advantage of this screening system is that i) neither the substance library nor the target protein need be labelled. As a result, a considerable rationalisation of the process can be achieved. In addition, the properties of the substances contained in the library and the protein are not altered by the labelling generally introduced subsequently in the conventional method. ii) If the screening is focussed on the active centre, then all substances binding to other areas of the protein are not detected. This increases the chances of identifying substances having the desired inhibitory effect.

The invention is explained in the following with reference to the drawings and examples from which further features, embodiments and advantages of the various aspects of the invention are obtained. In the figures

FIG. 1 is a schematic diagram of the activation/inactivation cycle of the ARF proteins,

FIG. 2 is a comparative overview of the family of ARF-GEF proteins,

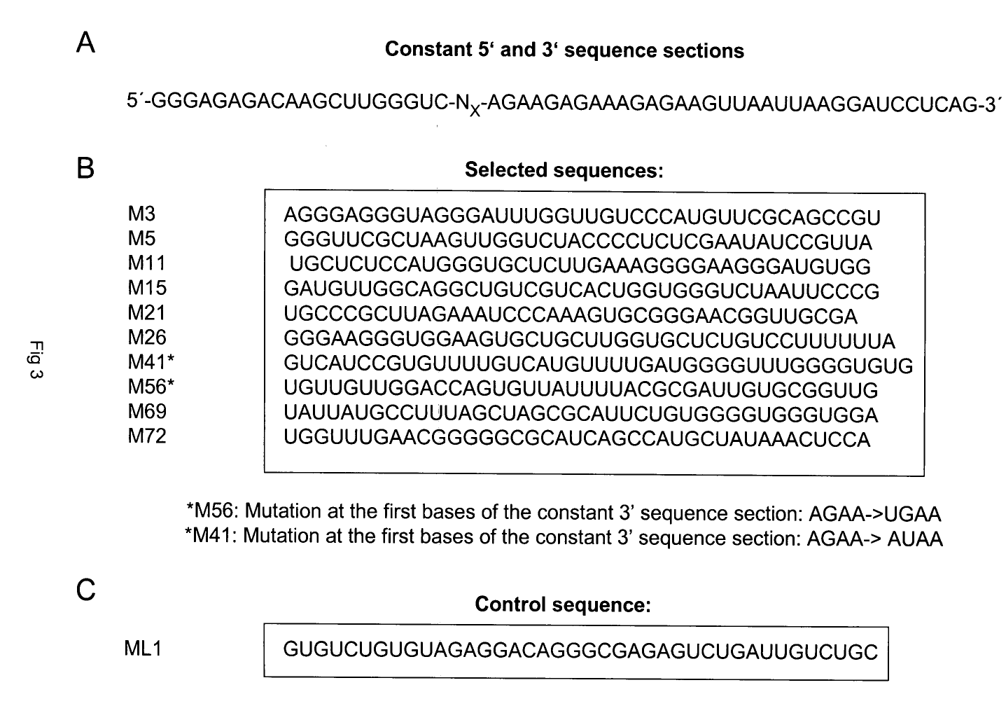

FIG. 3 shows the sequences of the RNA aptamers found in the in vitro selection experiment against cytohesin-1,

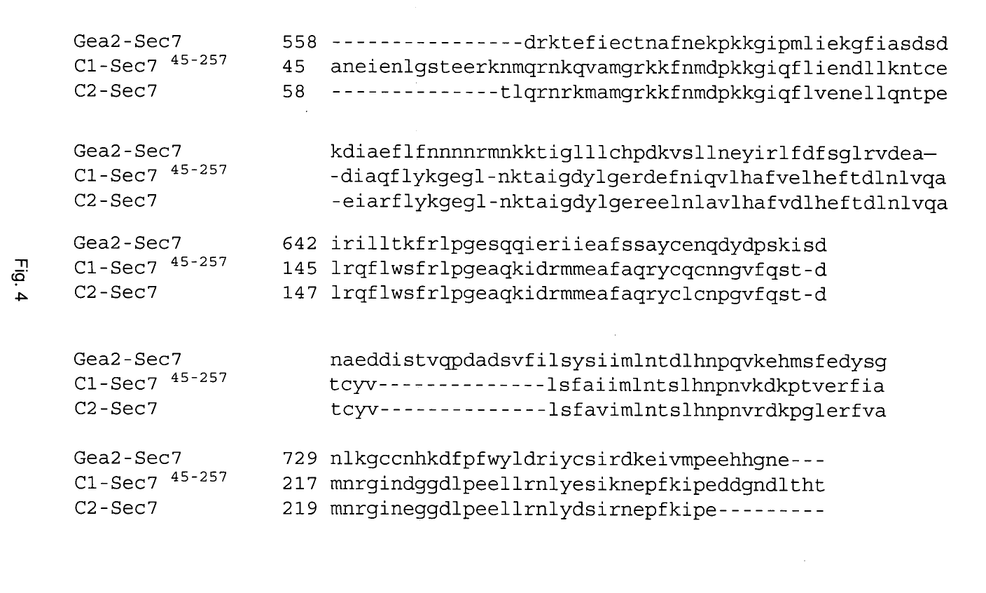

FIG. 4 shows a comparison of the amino acid sequence of the Sec-7 domains of Gea2, cytohesin-1 and cytohesin-2,

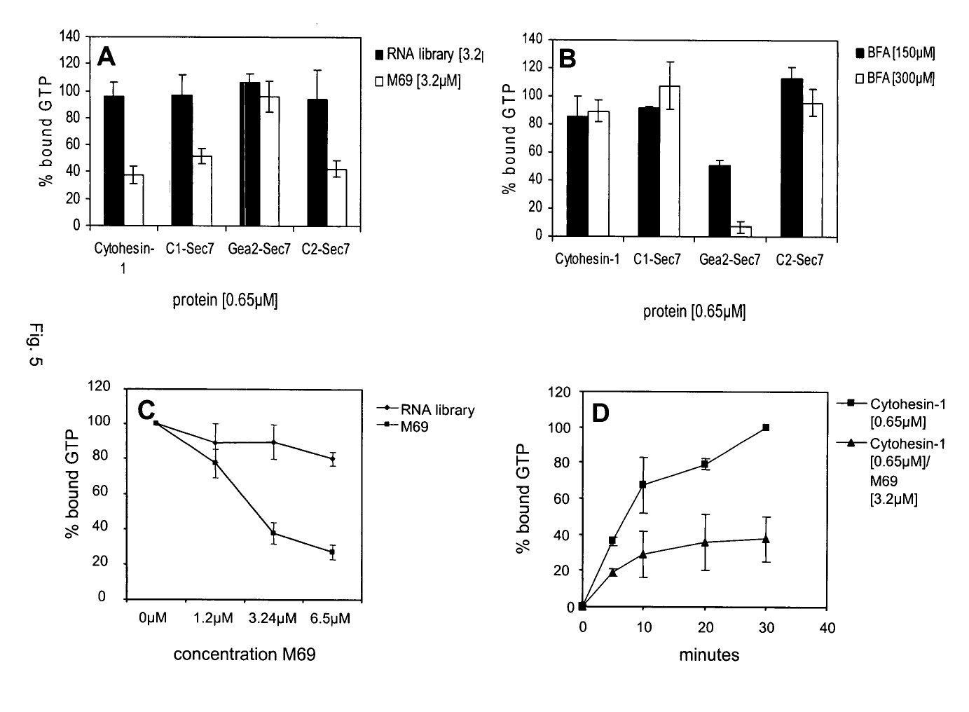

FIG. 5 shows the inhibition of the GDP/GTP exchange activity by one of the nucleic acid sequences according to the invention (aptamer M69),

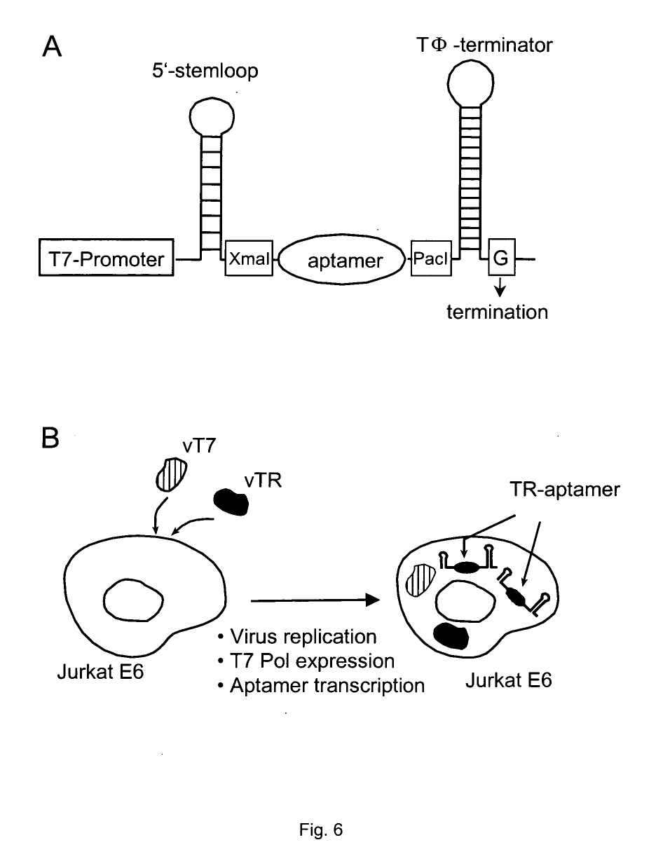

FIG. 6 shows the TR expression system for the expression of intramers (intracellular aptamers)

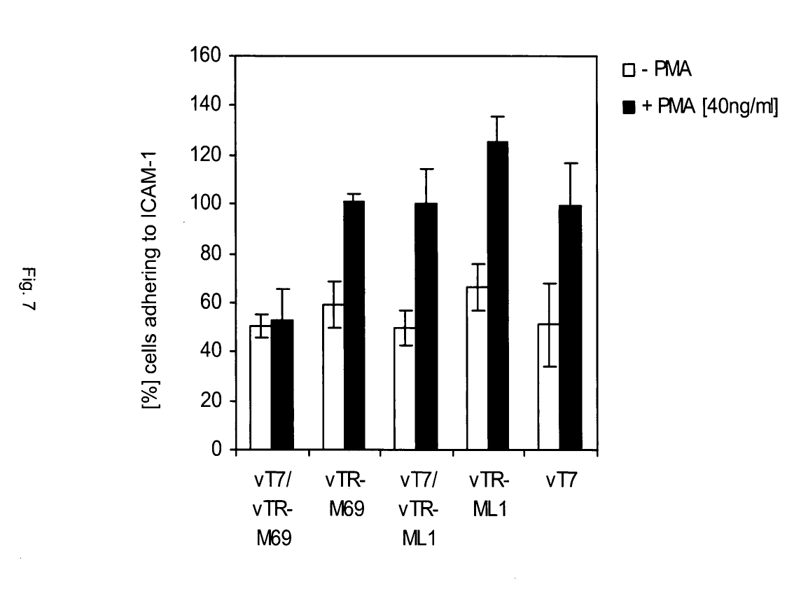

FIG. 7 shows the inhibition of the adhesion of Jurkat E6 cells by their LFA-1 integrins on ICAM-1, when the aptamer M69 is expressed by the TR expression system in the cells

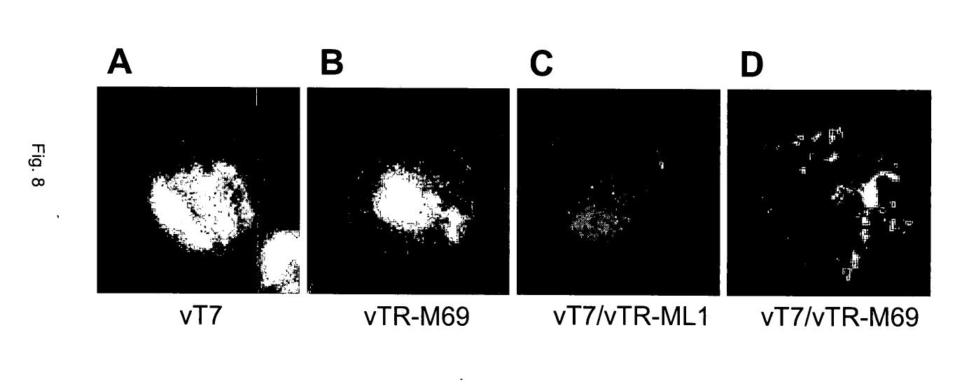

FIG. 8 shows the fluorescence staining of the actin cytoskeleton following expression of the intramer TR-M69 and various controls,

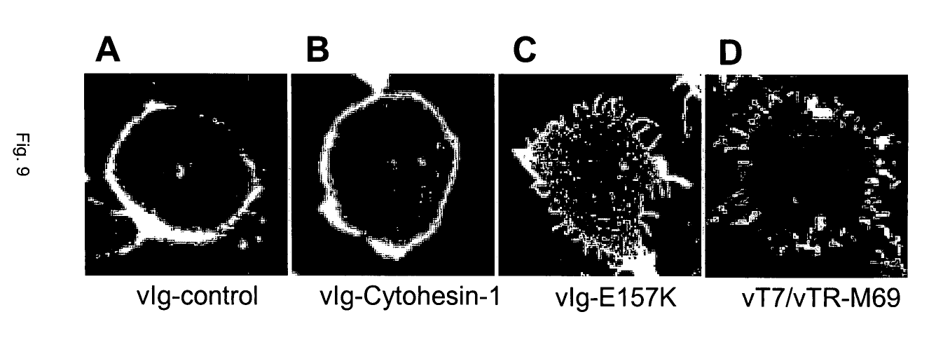

FIG. 9 shows the fluorescence staining of the actin cytoskeleton following expression of the intramer TR-M69 and various control proteins;

FIG. 10 shows the principle of the manufacture of an aptazyme based on one of the nucleic acids according to the invention;

FIG. 11 shows a pol III expression cassette; and

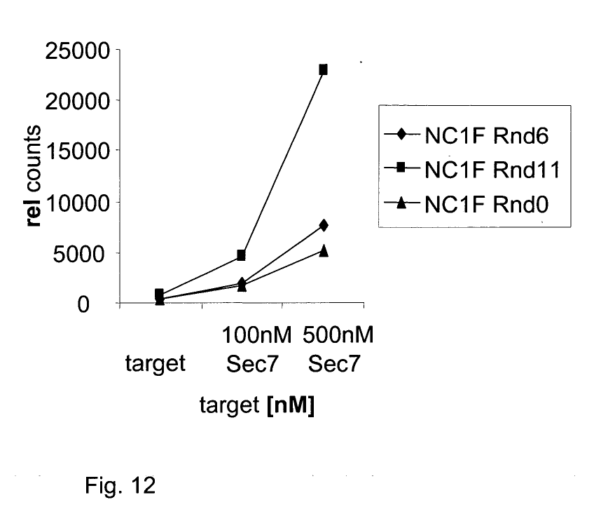

FIG. 12 shows the result of a study on the binding of unselected and selected nucleic acid pool to the Sec-7 domain of cytohesin-1.

FIG. 1 shows a schematic diagram of the activation and deactivation cycle of ARF proteins. ARF proteins belong to a family of regulatory GTPases which are also designated as G proteins. ARF proteins are involved in regulating the membrane organisation in eukaryotic cells and are found both in single-cell organisms such as yeasts and also in higher eukaryonts such as plants and animals. If GDP is bound to the ARF proteins, these are inactive. One group of proteins, the so-called GEF (“guanosine nucleotide exchange factors”) proteins catalyse the exchange of GDP for GTP and thus activate the ARF proteins. The groups of GEF proteins specific for ARF proteins are herein designated as ARF-GEF proteins. Another group of proteins, the so-called GAP (“GTPase activating protein”) proteins induce the GTPase activity of the ARF proteins. After the release of orthophosphate (Pi), the ARF proteins are again in the GDP-bound, inactive state.

FIG. 2 shows an overview of the various members of the ARF-GEF proteins (according to Donaldson and Jackson, Curr. Opin. Struct. Biol. 12 (2000), 475-482). The black box represents the Sec7 domain for which the GDP/GTP exchange activity is responsible. The DCB domain (dotted) mediates the and the binding of cyclophillins. The CC (coiled coil) and PH (pleckstrin homology) domains of the small ARF-GEFs are shown oblique and longitudinally striped. EFA6 is a new protein which is different from cytohesin. It belongs to the family of ARF-GEF proteins but neither to the group of large or the group of small ARF-GEF's (such as cytohesin 1, 2, 3 and 4). However, it is Brefeldin A-sensitive, has a molecular weight of around 70 kD and additionally contains prolin-rich motifs. The sensitivity or resistance to Brefeldin A is given.

FIG. 3 shows the basic structure of the library of nucleic acid sequences which was used for the in vitro selection and the sequences of the aptamers obtained by the in vitro selection against cytohesin-1. a) shows the 5′ and 3′ constant sections of the RNA libraries which flank the individual sequences of the RNA library (shown by Nₓ). b) shows the individual sequences corresponding to the region Nₓ of the aptamers from the in vitro selection against cytohesin-1. For the sequences M56 and M41 (marked by *) point mutations are given in the first bases of the 3′ constant region which were identified during the sequencing of the enriched RNA library from cycle 11. c) shows the sequence ML1 which does not bind cytohesin-1 and was used as a negative control in further experiments analogous to the sequences form b). The sequence ML1 can also be used as a negative control in the kits described herein.

FIG. 4 shows a comparison of the amino acid sequence (given in single letters) of the Sec7 domains of the high-molecular ARF-GEFs Gea2 (Gea2-Sec7) (Brefeldin A sensitive), and the small ARF-GEFs cytohesin-1 (C1-Sec7) and cytohesin-2 (C2-Sec7) (both not Brefeldin A sensitive), which are targets of the nucleic acids according to the invention. The amino acid positions are given.

FIG. 5 shows the data for the in vitro inhibition of the GDP/GTP exchange activity with ARF-1 as substrate, mediated by one of the nucleic acids according to the invention which was used as an aptamer in the experiments shown in this figure. A: GDP/GTP exchange activity of cytohesin-1 (full-length protein) C1-Sec7, C2-Sec7 and Gea2-Sec7 (see FIG. 4) with ARF-1 as substrate in the presence of the aptamer M69 (3.2 μM) (white bars) and the non-selected RNA library (3.2 μM) (black bars). The quantity of γ³⁵S-GTP bound to ARF-1 after 30 minutes in an experiment using cytohesin-1 without RNA was set as 100%. It can be clearly seen that the quantity of γ³⁵S-GTP, which is bound to ARF-1 with the small ARF-GEF cytohesin-1 or the Sec7 domains C1-Sec7 and C2-Sec7 in the presence of the aptamer M69 is reduced compared with the non-specific control (white bars). On the other hand, the binding of γ³⁵S-GTP to ARF-1 catalysed by the Sec7 domains of the high-molecular ARF-GEF Gea2 (Gea2-Sec7) is not influenced. B: Sensitivity of the GDP/GTP exchange of cytohesin-1, C1-Sec7, C2-Sec7 and Gea2-Sec7 towards Brefeldin A. Whereas the Sec7 domains of the high-molecular ARF-GEF's (Gea2-Sec7) are clearly blocked by Brefeldin A, as predicted this inhibitor has no influence on cytohesin-1, C1-Sec7 or C2-Sec7. C: Concentration dependence of the inhibition of the GDP/GTP exchange activity of cytohesin-1 on ARF-1 by the aptamer M69 (black squares) and the non-selected RNA library (black rhombs). The concentration dependence of the concentration of the aptamer inhibitor M69 can be clearly seen whereas increasing concentrations of the non-specific RNA library have little influence on the quantity of γ³⁵S-GTP bound to ARF-1. D: Time behaviour of the inhibition of the cytohesin-1-mediated GDP/GTP exchange activity with aptamer inhibitor M69 (black triangles) and without any aptamer (black squares).

FIG. 6 shows the TR expression system for the cytoplasmic expression of intracellular aptamers (intramers) by means of recombinant vaccinia viruses. This system is described in detail in Blind et al., Proc. Natl. Acad. Sci. USA 96 (1999), 3606-3609 and Blind et al., PCT/EP/00/02727) A: The TR expression cassette. The aptamer is cloned via the restriction sites Xma I and Pac I into the expression cassette which is under the control of the T7-RNA polymerase promoter. In the transcript the aptamer-RNA is flanked by two stem-loop structures (5′-stem loop and Tφ-terminator). These additional RNA sequences are used for intracellular stabilisation and the correct termination of the aptamer by the T7-RNA-polymerase. The aptamer sequences in the TR expression context are designated as TR intramers. B: Infection scheme for expression of the TR intramers. For expression of the TR intramers the target cells (in this case Jurkat-E6 cells, TIB 152, American Type Culture Collection (ATCC), Manassas, USA)) were infected with two recombinant vaccinia viruses. The one (vTR) carries the TR expression cassette in its genome and the other (vT7) expresses the T7-RNA-polymerase under the control of a vaccinia promoter. After infection of the cells with the two viruses, the TR expression cassettes are transcribed by the formed T7-RNA-Polymerase. Since the life cycle of the vaccinia viruses takes place exclusively in the cytoplasm, the TR intramer RNA will also be expressed there.

FIG. 7 shows the inhibition of the cellular adhesion mediated by the integrin LFA-1 when the intramer TR-M69 is expressed in Jurkat E6 cells (see FIG. 6). The adhesion of Jurkat E6 cells to Petri dishes which were coated with the natural interactor of the LFA-1 integrin ICAM-1 (“intercellular adhesion molecule-1”) can be stimulated by the addition of phorbol esters (PMA) (stimulated values: black bars; unstimulated values: white bars). The stimulation can be readily observed during single infection with various control vaccinia viruses (vT7, vTR-ML1, vTR-M69). In the same way, the expression of a control sequence (ML1, see FIG. 3c) by double infection with the recombinant vaccinia viruses vT7 and vTR-ML1 (vT7/vTR-ML1) has no effect on the stimulated adhesion of the cells. The value for the stimulated adhesion of cells which were only infected with the recombinant virus vTR-M69 for control was taken as the 100% value. However, if the aptamer TR-M69 specific for the Sec7 domain of cytohesin-1 is expressed by double infection with the virus vT7 and vTR-M69 (vT7/vTR-M69) in Jurkat E6 cells, the PMA-induced stimulated adhesion is completely blocked.

FIG. 8 shows the fluorescence staining of the actin cytoskeleton after infection of Jurkat E6 cells with recombinant vaccinia viruses. Five hours after infection with the various recombinant vaccinia viruses described below, the Jurkat E6 cells (TIB 152, American Type Culture Collection (ATCC), Manassas, USA) were stimulated with 40 ng/ml of PMA for half an hour at 37° C. and adhered to the fibronectin-coated slides. After fixing and permeabilisation, the actin cytoskeleton was stained with TRITS-labelled phalloidin. The cells were visualised by fluorescence microscopy. A: single infection of the cells with a recombinant vaccinia virus which expresses the T7-RNA polymerase (vT7). The expression of the polymerase alone results in a normal cellular morphology and organisation of the cytoskeleton stained with TRITS phalloidin. B: infection of the cells with a recombinant vaccinia virus which carries the expression cassette for the intracellular aptamer (intramer) TR-M69 integrated in its genome. As a result of the lack of a suitable polymerase, in this case a T7 polymerase, the TR intramer is not expressed and the infection does not interfere with the organisation of the cytoskeleton. C: double infection of the cells with the recombinant vaccinia viruses vT7 and vTR-ML 1. The expression of the control sequence TR-ML1 by the double infection with vT7 and vTR-ML 1 has no influence on the organisation of the cytoskeleton. D: the expression of the aptamer TR-M69 by double infection with the recombinant vaccinia viruses vT7 and vTR-M69 disturbs the structuring of the actin cytoskeleton and brings about a modified star-shaped morphology.

FIG. 9 shows the fluorescence staining of the actin cytoskeleton following infection of Jurkat E6 cells with recombinant vaccinia viruses. The cells stained with TRITS phalloidin and adhering to fibronectin after stimulation with PMA were examined using confocal laser microscopy. A horizontal section of the cells analysed in each case is shown. A: infection of the cells with a control virus which expresses recombinant CH1-CH2 domains of the human IgG immunoglobulin. The expression of the IgG fragment has no effect on the morphology of the cells. B: infection of the cells with a recombinant vaccinia virus which expresses human cytohesin-1 as fusion protein with the CH1-CH2 domains of the human IgG immunoglobulin. The expression of cytohesin-1 does not interfere with the organisation of the cytoskeleton. C: infection of the cells with a recombinant vaccinia virus which expresses the mutated form of cytohesin-1 (E157K) where glutamate is exchanged in position for lysin, whereby the mutant loses the GDP/GTP exchange activity, as fusion protein with the CH1-CH2 domains of the human IgG immunoglobulin. Infection with vE157K leads to a significant change in the organisation of the actin cytoskeleton. D: the expression of the aptamer TR-M69 by double infection with the recombinant vaccinia viruses vT7 and vTR-M69 brings about the same phenotypic changes as the expression of the mutant form of cytohesin-1 (vE157K) (FIG. 9c).

FIG. 10 shows the principle of manufacturing an aptazyme on the basis of one of the nucleic acids according to the invention and the detection of the binding of the Sec7 domain to the aptazyme construct. A) The aptazyme consists of an allosteric centre which consists of one of the nucleic acids according to the invention (aptamer domains). This allosteric centre is connected via a binding member sequence to the ribozyme domain. In this case, the ribozyme domain comprises the catalytic centre of a so-called hammerhead ribozyme (HHR). The substrate for the aptazyme is a so-called FRET oligonucleotide (substrate: underlined letters) which is labelled at the 5′ end with the fluorescent dye FAM (donor: 6-carboxy fluorescein) and at the 3′ end with the fluorescent dye TAMRA (quencher: 6-carboxy-tetramethyl rhodamine). Such oligonucleotides can be produced synthetically and are available commercially (e.g. through Eurogentec, Seraign, Belgium or IBA, Göttingen, Germany). The FRET-oligonucleotide binds to the ribozyme domain via complementary base pairing. According to the principle commonly used for FRET assays which is familiar to the person skilled in the art (Hanne et al., Nucleosides and Nucleotides 17 (1998), 1835-1850; Singh et al., RNA 5, 1348-1356). As a result of the spatial closeness of the donor to the quencher, the fluorescence emission of the donor (hv′: 516 nm) can be absorbed by the quencher following excitation at its absorption wavelength (hv: 496 nm). This effect is based on the fact that the emission wavelength of the donor overlaps with the absorption wavelength of the quencher. No measurable signal appears. In the form shown here the aptazyme is inactive. B) If the Sec7 domain binds to the aptamer domain (allosteric centre) of the aptazyme, this induces a conformational change which results in activation of the ribozyme domain and cleaving of the substrate. After hydrolysis of the FRET substrate by the ribozyme, the cleaved fragments can be removed from one another in solution. The fluorescence (emission: hv′) of the donor is now no longer intramolecularly quenched by the quencher and the ribozyme is available for a next catalysis cycle. The clearing of the FRET effect brings about a measurable increase in the FAM-specific fluorescence in the sample. This effect is known to the person skilled in the art and is described in the literature (Jenne A. et al., Angew. Chem. 111 (1999), 1383-1386). Since the increase in the fluorescence is directly proportional to the cleaving activity of the ribozyme, the binding of the Sec7 domain to the aptazyme can be detected by the measured data. The sequences of the substrate and the ribozyme domain shown here are given as non-limiting examples. In principle, other constructs of ribozyme domains and substrates or methods of detecting the ribozyme activity known to the persons skilled in the art can be used (review article: Soukup and Breaker, Curr. Opin. Struct. Biol. 10 (2000), Soukup and Breaker, Trends Biotechnol. 17 (1999), 469-76)

### EXAMPLE 1

### Selection of Cytohesin-Binding Aptamers

RNA aptamers against cytohesin-1 were selected by in vitro selection from a library of RNA molecules with a randomised region of 40 nucleotides. The randomised region was flanked on the 5′ and 3′ sides by 2 constant regions which during the selection process served as primer annealing sequences for the enzymatic amplification of the nucleic acids by reverse transcription and the polymerase chain reaction (see also FIG. 3). In order to generate the nucleic acid library for the in vitro selection an ssDNA oligonucleotide having the sequence 5′-CTATA GGGAG AGACA AGCTT GGGTC N₄₀ AGAAG AGAAA GAGAA GTTAA TTAAG GATCC TCAG-3′ (herein also designated as SEQ ID No. 14) (N₄₀ stands for 40 randomised positions with a balanced ratio of the four bases A,C,G and T of 1:1:1:1) was synthesised on an oligonucleotide synthesis apparatus (Expedite, Millipore) using the phosphoramidite method. The single-stranded oligonucleotide was made double-stranded by PCR with the primers 5′-TCT AAT ACG ACT CAC TAT AGG GAG AGA CAA GCT TGG GTC-3′ (in accordance with SEQ ID No. 12) and 5′-CTG AGG ATC CTT AAT TAA CTT CTC TTT CTC-3′ (in accordance with SEQ ID No. 13). The complexity of the RNA library was around 10¹⁵ different sequences.

The fundamental steps of the selection process are described in detail in the literature and are known to the person skilled in the art (Tuerk, C. and Gold, L., Science 249 (1990), 505-510; Ellington, A. D. and Szostak, J. W., Nature 346 (1990), 818-822; Nieuwlandt, D. et al., Biochemistry 34 (1995), 5651-5659; Conrad, R. et al. Methods Enzymol. 267 (1996)). The cytohesin-1 protein was provided as fusion protein with a consecutive sequence of six histidine residues, also designated as 6× his-tag (cytohesin-1-his6), overexpressed in E. coli and purified by Ni²⁺-NTA affinity chromatography. The protocols for the expression and purification of his-tag proteins are established and known to the persons skilled in the art (Kolanus et al., Cell 86 (1996), 233-242). 3.75 mg of the purified cytohesin-1 protein was coupled to CNBr-activated sepharose according to the information given by the manufacturer (Amersham Pharmacia Biotech). As preselection templates, on the one hand CNBR-activated sepharose was blocked with 0.2 M of glycin and on the other hand, lysates of E. coli cells which were treated by analogy with cytohesin-1-His6. Washing and elution fractions of the pure E. coli lysates on the Ni2+-NTA affinity template were respectively coupled to CNBr-activated sepharose (Pharmacia) and used in the subsequent preselection. In order to remove non-bound proteins, all prepared sepharoses were intensively washed with buffer (137 mM NaCl, 2.7 mM KCl, 4.3 mM Na₂HPO₄*7H₂O, 1.4 mM KH₂PO₄) and stored at 4

For the first selection round 1.4 nmol of the dsDNA library described above was used as template for the in vitro transcription reaction with T7-RNA-polymerase (Stratagene) according to the information given by the manufacturer. For all following selection cycles 250 pmol of DNA was used as template. The in vitro transcribed RNA was purified by electrophoresis on 8% denaturing polyacrylamide gels. The nucleic acid bands were visualised by fluorescence quenching. For this purpose the preparative polyacrylamide gels were packed in transparent film, placed on a thin-layer chromatographic plate with fluorescence indicator (DC-Alufolie Kieselgel 60 F₂₅₄, Merck) and irradiated with a hand-held UV light at a wavelength of 254 nm. Nucleic acid bands of the correct length were excised using a sterile scalpel and eluted by the “crush and soak” method in 0.3M sodium acetate. After precipitation of the nucleic acids, the RNA was taken up in sterile H₂O. For the selection the RNA library was diluted in different concentrations (see Table 1) in selection buffer (3 mM MgCl₂, 137 mM NaCl, 2.7 mM KCl, 4.3 mM Na₂HPO₄*7H₂O, 1.4 mM KH DTT, RNasin 0.8 U/μL, pH 7.3) and denatured for 1 minute at 95° C. For RNA folding the library was incubated for 10 min at 4° C. The protein sepharoses were then added. The precise reaction conditions are given in the following Table 1.

TABLE 1
Parameters of the thirteen cycles of the in vitro selection experiment against cytohesin-1.

| Selection cycle | Column vol. | Preselection | RNA (pmole) | Incubation vol. | Wash vol. | Elution vol. | Kompetitor | Selection cycle |
|---|---|---|---|---|---|---|---|---|
| 1 | 100 | — | 2000 | 2000 | 1000 | 1500 (Urea/EDTA) | — | 199 |
| 2 | 50 | Glycin/100 | 1000 | 412 | 600 | 400(U/E)/200 (ELU) | — | 98 |
| 3 | 25 | Glycin/50 | 500 | 466 | 800 | 400 (ELU) | — | 47 |
| 4 | 25 | E.coli E/50 | 500 | 466 | 900 | 400 (ELU) | — | 46 |
| 5 | 25 | E.coli W/50 | 500 | 466 | 900 | 400 (ELU) | — | 45 |
| 6 | 25 | E.coli W/50 | 500 | 466 | 900 | 400 (ELU) | — | 44 |
| 7 | 25 | E.coli W/25 | 500 | 466 | 900 | 600 (ELU) | — | 43 |
| 8 | 25 | E.coli W/25 | 250 | 466 | 900 | 400 (ELU) | — | 42 |
| 9 | 25 | E.coli E/25 | 250 | 466 | 1000 | 400 (ELU) | — | 41 |
| 10 | 25 | E.coli E/25 | 250 | 466 | 1000 | 400 (ELU) | — | 40 |
| 11 | 25 | E.coli E/25 | 250 | 170 | 1600 | 600 (ELU) | 2.5 ug/ul Heparin | 39 |
| 12 | 25 | E.coli E/25 | 250 | 466 | 47000 | 400 (ELU) | — | 38 |
| 13 | 5 | E.coli E/25 | 250 | 423 | 8400 | 400 (ELU) | 1.2 ug/ul Hepariin | −3 |

The binding reactions took place at 37° C. for 1 h. The sepharose was then washed intensively with selection buffer to remove all non-bound RNA sequences. Bound RNA molecules were isolated by washing with denaturing buffer (30 mM tris pH 6.8, 20% glycerin, 2% SDS, 1M DTT), extracted with phenol and chloroform and precipitated by ethanol precipitation. The RNA was reverse transcribed with Tth DNA polymerase according to the information given by the manufacturer (Roche) and amplified with DAp DNA polymerase (Eurogentec) by polymerase chain reaction (PCR). The PCR-DNA served as template for the following in vitro transcription. Preselections were carried out beginning with the second selection cycle either using glycin sepharose or E. coli lysate sepharose. From cycles 11 to 13 heparin in a concentration of 1 μg/ml was added to the selection buffer to increase the stringency. After the thirteenth selection round the nucleic acid library was cloned and 72 sequences analysed. Among the 72 isolates ten different sequences could be identified (see FIG. 3). The most frequently found sequences were the clones M69 (61%) and M3 (18%). The remaining sequences were represented with a lower frequency. The sequences of these ten clones correspond to SEQ ID No. 1 to 10 and were ascribed herein as follows:

SEQ ID No. 1, herein also designated as M3:
5′-GGGAGAGACAAGCUUGGGUCAGGGAGGGUAGGGAUUUGGUUGUCCCAUGUUCGCAGCCGUA
GAAGAGAAAGAGAAGUUAAUUAAGGAUCCUCAG-3′
SEQ ID No. 2, herein also designated as M5:
5′-GGGAGAGACAAGCUUGGGUCGGGUUCGCUAAGUUGGUCUACCCCUCUCGAAUAUCCGUUAAG
AAGAGAAAGAGAAGUUAAUUAAGGAUCCUCAG-3′
SEQ ID No. 3, herein also designated as M11:
5′-GGGAGAGACAAGCUUGGGUCUGCUCUCCAUGGGUGCUCUUGAAAGGGGAAGGGAUGUGGAGA
AGAGAAAGAGAAGUUAAUUAAGGAUCCUCAG-3′
SEQ ID No. 4, herein also designated as M15:
5′-GGGAGAGACAAGCUUGGGUCGAUGUUGGCAGGCUGUCGUCACUGGUGGGUCUAAUUCCCGAG
AAGAGAAAGAGAAGUUAAUUAAGGAUCCUCAG-3′
SEQ ID No. 5, herein also designated as M21:
5′-GGGAGAGACAAGCUUGGGUCUGCCCGCUUAGAAAUCCCAAAGUGCGGGAACGGUUGCGAAGA
AGAGAAAGAGAAGUUAAUUAAGGAUCCUCAG-3′
SEQ ID No. 6, herein also designated as M26:
5′-GGGAGAGACAAGCUUGGGUCGGGAAGGGUGGAAGUGCUGCUUGGUGCUCUGUCCUUUUUUAA
GAAGAGAAAGAGAAGUUAAUUAAGGAUCCUCAG-3′
SEQ ID No. 7, herein also designated as M41:
5′-GGGAGAGACAAGCUUGGGUCGUCAUCCGUGUUUUGUCAUGUUUUGAUGGGGUUUGGGGUGU
GAUAAGAGAAAGAGAAGUUAAUUAAGGAUCCUCAG-3′
SEQ ID No. 8, herein also designated as M56:
5′-GGGAGAGACAAGCUUGGGUCUGUUGUUGGACCAGUGUUAUUUUACGCGAUUGUGCGGUUGUG
AAGAGAAAGAGAAGUUAAUUAAGGAUCCUCAG-3′
SEQ ID No. 9, herein also designated as M69:
5′-GGGAGAGACAAGCUUGGGUCUAUUAUGCCUUUAGCUAGCGCAUUCUGUGGGGUGGGUGGAAG
AAGAGAAAGAGAAGUUAAUUAAGGAUCCUCAG-3′
SEQ ID No. 10, herein also designated as M72:
5′-GGGAGAGACAAGCUUGGGUCUGGUUUGAACGGGGGCGCAUCAGCCAUGCUAUAAACUCCA
AGAAGAGAAAGAGAAGUUAAUUAAGGAUCCUCAG-3′
Control sequence, herein also designated as SEQ ID No. 11 or ML1.
5′-GGGAGAGACAAGCUUGGGUCGUGUCUGUGUAGAGGACAGGGCGAGAGUCUGAUUGUCUGCAG
AAGAGAAAGAGAAGUUAAUUAAGGAUCCUCAG-3′

### EXAMPLE 2

### Binding Specificities of the Selected Aptamers

Filter binding experiments were carried out in order to test whether the selected aptamer sequences can also recognise native cytohesin-1 in solution. The interaction analyses are described in detail in the literature. (Jellinek et al., Proc. Natl. Acad. Sci. USA 90 (1993), 11227-11231; Jellinek et al., Biochemistry 33 (1994), 10450-10456).

Increasing concentrations of cytohesin-1-His6 (0.1 nM to 500 nM) were incubated with the aptamers according to the invention (1.0 nM) radioactively labelled with ³²P at the 5′ end for 1 h at 37° C. in selection buffer. The RNA/cytohesin-1-His6 complexes were then filtered over nitrocellulose acetate mixed membranes (HAWP 0.45 μm, Millipore). The filter was then rinsed with 5 ml of washing buffer (3 mM MgCl₂, 137 mM NaCl, 2.7 mM KCl, 4.3 mM Na₂HPO₄.7H₂O, 1.4 mM KH₂PO₄). Before the experiment the filters were pretreated for 10 minutes with 40 mM 6-aminocaproic acid and 20% ethanol. Radioactive RNA retained on the filters was quantified by analysis using a phosphor imager (Storm 860, Amersham). The dissociation constant (K_{d}) was determined as described by Jellinek et al. (Jellinek et al., Proc. Natl. Acad. Sci. USA 90 (1993), 11227-11231). The value of K_{d} for the non-selected RNA library determined for comparison for the cytohesin-1-His6-protein was >4 μM. Of the analysed sequences the clone M69 bound cytohesin-1-His6 with a dissociation constant of 16 nM (see Table 2). As controls the same binding experiments were carried out with E. coli lysates which were treated similarly to the cytohesin-1 expressed in E. coli. The four sequences tested (M56, M41, M5, M3) also showed binding to cytohesin-1.

In the next step the binding site of the aptamer M69 on cytohesin-1 was delimited in more detail. For this purpose filter binding studies were carried out using subdomain constructs of cytohesin-1. The PH domain and the Sec7 domain of cytohesin-1 were overexpressed as 6×His-Tag proteins (PH-His6 and C1-Sec7-His6) in E. coli and purified as described for cytohesin-1-His6. The synthetic polybasic C-peptide was obtained from the company TopLab (Munich). The Sec7 domain of the large Brefeldin A-sensitive ARF-GEF's Gea2 (Gea2-Sec7) was made available as 6×His-Tag protein by Cathy Jackson (Departement de Biologie Cellulaire et Moleculaire, CEA/Saclay, France). The binding studies showed that the C1-Sec7 domain like the full-length protein was bound by M69 with a K_{d} value of 16 nM. On the other hand, no interaction with the PH domain or the Sec7 domain of Gea2 could be detected (see Table 2). The experiments show that the amino acid sequence of the Sec7 domain of the small ARF-GEF's cytohesin-1 is required for binding of the aptamer M69 and that the aptamer C1-Sec7 can differ very specifically from the related Sec7 domain of the large ARF-GEF's Gea2. The aptamer M5 on the other hand recognises both the Sec7 domain of cytohesin-1 and also of Gea2 with approximately the same affinity. The PH domain of cytohesin-1 is recognised an order of magnitude less well. The aptamer M56 however binds best to the full-length protein cytohesin-1 and barely to the Sec7 domain of Gea2. Since both the Sec7 domain and also the PH domain of cytohesin-1 are bound with somewhat reduced affinities compared with the full-length protein, the aptamer appears to recognise a binding site on cytosin-1 which is composed of both domains. The Sec7 domains of Gea2, cytohesin-1 (C1) and cytohesin-2 (C2) are shown in FIG. 4.

TABLE 2
Affinities of several aptamers (M5, M56, M69) from the
selection against cytohesin-1. The dissociation constant (nM)
was determined for cytohesin-1 (full-length protein), the Sec7
domain of cytohesin-1 (C1-Sec7), the PH domain of
cytohesin-1 (C1-PH) and the Sec7 domain of Gea2
(G2-Sec7). If no interaction was measurable, this is
given by (—). All the data on the dissociation constant
K_{d} are given in nM.

| | Cytohesin-1 | C1-Sec7 | C1-PH | G2-Sec7 |
|---|---|---|---|---|
| Aptamer M5 | 40.0 ± 8.0 | 36.0 ± 3.4 | 480 ± 50 | 48.0 ± 5.5 |
| M56 | 5.0 ± 1.0 | 280 ± 28 | 150 ± 8.0 | >1000 |
| M69 | 16.0 ± 0.4 | 16.0 ± 0.3 | — | — |

### EXAMPLE 3

### Specific Inhibition of the Guanine Nucleotide Exchange on ARF-1

The exchange of GDP for GTP activates the ADP ribosylation factors (ARF's). Since it has been shown that the aptamer M69 only specifically recognises the Sec7 domain of the small ARF-GEF's, the influence of the aptamer on its GDP/GTP exchange function was studied. For this purpose the effect of the RNA molecule M69 on the exchange of GDP for GTP on the ARF1 protein was investigated in vitro in the presence of radioactive [γ³⁵S]-GTP.

A chimera ARF-1 protein to which the constant CH1 and CH2 domains of the human IgG immunoglobulin were fusioned at the carboxy terminus for purification purposes was overexpressed in COS or CV-1 cells by infection with recombinant vaccinia viruses (Knorr et al., Eur. J. Biochem. 267 (2000), 3784-3791). The cells were harvested with a cell scraper and washed twice with PBS. The cells were then solubilised with lysis buffer (100 mM tris (pH 8,0), 150 mM NaCl, 2 mM EDTA, 1% (v/v) Triton X-100, 10 μg/mL leupeptin, 1 mM phenylmethylsulfonylfluoride) and centrifuged at 20 000 g and 4° C. for 4 min. The supernatant was incubated with protein A-Sepharose 6 MB (Amersham Pharmacia Biotech) for 2 h at 4° C. The sepharose was washed twice with 1 ml of lysis buffer and equilibrated in 50 mM of HEPES. After completely removing the supernatant, the sepharose was equilibrated in buffer E (50 mM HEPES (pH 7.4), 1 mM MgCl₂, 1 mM DTT, RNasin 0.8 U/μl). Before adding ARF-1 cytohesin-1, C2-Sec7 or Gea2-Sec7 were incubated with renatured aptamer-RNA at 37° C. in buffer E. The exchange reaction was initiated by adding 0.02 μCi/μl [γ³⁵S]-GTP (NEN Life Sciences). After incubating at 37° C. for 30 min the reaction was stopped by adding 1 ml of washing buffer (20 mM HEPES pH 7.4, 100 mM NaCl, 10 mM MgCl₂), the protein A sepharose was washed twice again with washing buffer. The washing buffer was completely removed and after adding scintillation liquid, the bound radioactivity (γ³⁵-S GTP) was determined by measuring using a scintillation counter. In the same way, all time profiles were determined and stopped at different time points. The quantity of bound [γ³⁵S]-GTP in the reactions without aptamer-RNA was set as 100%. Attempts in which the non-selected RNA library was added, which had no effect on the ARF-GEF activity of all the GEF's studied (FIGS. 5a and c) were used as negative control. Cytohesin-1 (0.65 μM) increased the binding of [γ³⁵S]-GTP to ARF-1 tenfold compared with the uncatalysed reaction (data not given). These results show good agreement with the values described in the literature (Knorr et al. Eur. J. Biochem. 267 (2000), 3784-3791). The experiments showed that the aptamer M69 is a potent inhibitor of the GTP/GDP exchange on ARF-1 by cytohesin-1 or the isolated cytohesin-1-Sec-7 domain (C1-Sec7). Furthermore, the exchange function of the Sec-7 domain (C2-Sec7) of cytohesin-2 (ARNO) is also inhibited (see FIG. 5a). The inhibition of cytohesin-1 is concentration-dependent (FIG. 5c). Moreover, the guanine nucleotide exchange catalysed by the Sec7 domain (G2-Sec7) of the large GEF-protein Gea2 is not influenced (see FIG. 5a). In control experiments with Brefeldin A it could be shown that as predicted, the small Sec7 proteins or domains are not inhibited although G2-Sec7 may be (see FIG. 5 b). The data show that the aptamer M69 according to the invention is specifically capable of distinguishing between the large and small GEF proteins and inhibiting the catalytic activity of the small GEF's. The aptamer is thus a specific inhibitor of the GDP/GTP exchange reaction of the Brefeldin A resistant small GEF proteins.

### EXAMPLE 4

### Inhibition of the Adhesion of Jurkat E6 Cells to ICAM-1

A system based on the infection of the target cells with recombinant vaccinia viruses was selected for the expression of the aptamer M69 according to the invention in the cytoplasm of Jurkat E6 cells (Blind et al., Proc Natl. Acad. Sci. USA 96 (1999), 3606-3610). In this connection the DNA coding for the aptamer is cloned via the restriction sites Xma1 and Pac1 into the TR expression cassette of the transfer vector pTR (see FIG. 6a). The aptamer-RNA transcribed by a T7-RNA-polymerase promoter is in this case flanked by a 5′ stem loop used for the cytoplasmic stabilisation and the Tφ-terminator used for the termination of the transcript. The transcripts thus formed are hereinafter called TR intramers—according to the invention (e.g. TR-M69). The recombinant vaccinia viruses were manufactured as described in the literature (Ausubel et al., Current Protocols in Molecular Biology (1987), Wiley & Sons Inc., New York, USA). In this connection the TR expression cassettes flanked in the transfer vector pTR by segments of the viral thymidine kinase gene (TK gene) are integrated into the TK gene of wild type vaccinia viruses by homologous recombination. The expression of the TR intramers was carried out using a double infection scheme described in the literature (Fuerst and Moss, J. Mol. Biol. 206 (1989), 333-348), which is shown in FIG. 6B. In this case, eukaryotic cells are infected on the one hand with the virus containing the gene for the TR intramer (vTR) and at the same time with a virus expressing the T7-RNA polymerase (vT7). After expression of the polymerase the TR intramers are then transcribed from the T7-RNA-polymerase promoter in the cytoplasm of the infected cells (Blind et al., Proc Natl. Acad. Sci. USA 96 (1999), 3606-3610). In addition to vaccinia viruses, other expression systems known to the persons skilled in the art such as Semliki Forest virus, retroviral vectors, plasmids, RNA-polymerase II and III controlled transcription units, among others can also be used for the expression of RNA-aptamers in cells (Cheetham G M, et al., Curr Opin Struct Biol. 10 (2000); 117-123; Paule and White, Nucleic Acid Res. 28 (2000), 1283-1298; Shi H. et al., Proc. Natl. Acad. Sci USA 96 (1999), 10033-10038; Bramlage et al., Tibtech 16 (1998), 434-438; Chakrabarti S, et al., Biotechniques. 23 (1997), 1094-1097; Tubulekas et al., Gene 190, 191-195; Bertrand et al., RNA 3 (1997), 75-88; Rossi, Tibtech 13 (1995), 301-305;.)

Five hours after the double infection of Jurkat E6 cells with the viruses vT7 and vTR-M69 (approx. 10 pfu/cell) expression levels of around 100 000 TR-intramer molecules per cell could be achieved. As already detected in earlier studes (Blind et al., Proc Natl. Acad. Sci. USA 96 (1999), 3606-3610), only the double infection with the virus (vT7) coding for the T7-RNA polymerase and the virus containing the expression cassette (vTR-intramer) results in transcription of the desired RNA molecules in the cytoplasm. In the case of infection with only one single recombinant vaccinia virus, no detectable quantities of transcribed RNA could be detected. All the experiments in Jurkat E6 cells were carried out in the following experiments five hours after infection with the recombinant vaccinia viruses.

The Sec7 domains of cytohesin-1 stimulate the adhesion of leukocytes both by the direct interaction with the β2-subunit of integrins and also by their ARF-GEF activity (Geiger, C. et al., EMBO J. 19 (2000), 2525-2536). Adhesion experiments have been carried out, as described earlier, in a leukocyte cell line (Jurkat E6 cells, TIB 152, American Type Culture Collection (ATCC), Manassas, USA), in which the activation of b2 integrins can be well reconstructed (Kolanus, W., et al., Cell 86 (1996), 233-242). 5 h after the infection with the recombinant viruses, the cells were incubated with 40 ng/ml of PMA (phorbol-12-myristate-13-acetate), a known inducer of β2-integrin activation, for 30 min at 37° C. The stimulated adhesion was detected as described earlier by incubating the cells on microtiter plates coated with the natural ligand (ICAM-1) of the β2-integrin LFA-1 (Nagel, W. et al., J. Biol. Chem. 273 (1998), 14853-14861).

As shown in FIG. 7, the adhesion of the cells could be significantly upregulated by adding PMA (black bars) as compared with the unstimulated background adhesion (white bars). All controls in which the Jurkat E6 cells were merely infected with one virus (vTR-M69, vTR-ML1, vT7) showed good stimulability. In the same way, the expression of a cytohesin-1 non-binding control sequence TR-ML1 (double infection: vT7/vTR-ML1) had no influence on the stimulability. On the other hand, the stimulated adhesion with expression of the Sec-7 domain binding aptamer TR-M69 (double infection: vT7/vTR-M69) is almost completely blocked (see FIG. 7).

As shown in earlier studies, a point mutant of cytohesin-1 (designated as E157K) can no longer catalyse the in vitro guanine nucleotide exchange on ARF proteins (Knorr, T. et al., Eur. J. Biochem. 267, 3784-3791). Overexpression of this mutant form in Jurkat E6 cells results in a strong inhibition of the cellular adhesion to ICAM-1. In agreement with these data, the experiments described above show that the aptamer M69 as an inhibitor of the ARF-GEF function in vitro can also block the adhesion in vivo. The aptamer is thus a first specific inhibitor which has been able to be developed and which is also active in cell culture against the small ARF-GEF proteins resistant to Brefeldin A.

### EXAMPLE 5

### TR-M69 and Cytohesin-1 (E 157K) Induced Changes to the Cellular Actin Cytoskeleton

Hitherto little has been known of the mechanistic role of the GEF activity of cytohesin-1 and also the in vivo substrate. Two possible functions have been discussed so far: transport of intracellular vesicles or restructuring of the actin cytoskeleton located on the cytoplasmic membrane. Earlier studies showed that the overexpression of cytohesin-1 in Jurkat E6 cells induced the propagation of the cells on ICAM-1-coated surfaces (Geiger, C. et al., EMBO J. 19 (2000), 2525-2536). However, this effect could not be observed when the mutant form of cytohesin-1 (E157K) was overexpressed. In order to better characterise the role of the ARF-GEF activity of cytohesin-1, the aptamer inhibitor TR-M69 according to the invention was now used and the effects on the restructuring of the actin cytoskeleton were studied.

For this purpose the Jurkat E6 cells as described were infected with the recombinant viruses and after 4.5 h were stimulated with 40 ng/ml of PMA for 30 min at 37° C. After centrifuging the cells and discarding the supernatant, the cells were taken up in HBSS (“Hanks buffered saline solution”) and applied to slides, which had been previously coated with fibronectin. For this purpose the slides were incubated for 2 h at room temperature in fibronectin solution and washed twice with HBSS. The Jurkat E6 cells were then left on the slides for 30 min at 37° C. and unbound cells were carefully washed with 2×100 μl HBSS. The adhering cells were then fixed on the slide with freshly prepared 2% formaldehyde (v/v) in PBS at 4° C. overnight. After incubating in 2% glycin (w/v) in PBS for 2 h, the cells were permeabilised with 0.2% Triton X-100 (v/v) for 10 min at room temperature. In order to visualise the actin cytoskeleton the cells were stained with TRITS-labelled phalloidin (Sigma-Aldrich Chemie GmbH, Deisenhofen, Germany) for 1 h at room temperature. The dye binds to actin filaments and is used for fluorescent representation of the actin cytoskeleton (Faulstich et al., Exp Cell Res 144, (1983), 73). The excess fluorescent dye was washed twice with 100 μl PBS and after applying “mounting medium” (Vector Laboratories, Burlingame, Calif.) the prepared cells were examined using a confocal laser microscope or a fluorescence microscope. Target cells which were simply infected with control virus (vT7 or vTR-M69) showed a normal adhesion phenotype (FIGS. 8a and b) which was also found with non-infected leukocytes (data not shown). In the same way the expression of the control sequence TR-ML1 by the double infection with the recombinant vaccinia viruses vT7 and vTR-M69 had no effect on the structure of the actin cytoskeleton (FIG. 8C). However, if the GEF-inhibitor TR-M69 was expressed in the Jurkat E6 cells (vT7/vTR-M69), the reorganisation of the cytoskeleton was significantly disturbed (FIGS. 8D and 9D).

The effect of the expression of the mutated cytohesin-1 (vlg-E157K) on the actin cytoskeleton was studied at the same time. As described earlier (Kolanus et al., Cell 86 (1996), 233-242) the overexpression of the proteins was achieved by recombinant vaccinia viruses. Cytohesin-1 and E157K were manufactured as fusion proteins with the constant CH2 and CH3 domains of the human IgG immunoglobulin. As shown in FIG. 9C, the overexpression of the mutant cytohesin-1 (vlg-E157K) induces the same phenotype as the expression of the aptamer TR-M69 according to the invention (FIG. 9D). The overexpression of cytohesin-1 (vlg-cytohesin-1) results in a normal phenotype (FIG. 9B). In the same way the expression of a control protein (vlg-control) had no detectable effect (FIG. 9a). The results clearly confirm that the in vitro selection process has produced specific inhibitors of the ARF-GEF activity of the small GEF proteins. In addition to the in vitro inhibition of the GEF function of cytohesin-1 and −2 (ARNO) the expression of the aptamer inhibited the adhesion of Jurkat E6 cells to ICAM-1 and changed the restructuring of the actin cytoskeleton with the adhesion to fibronectin. Thus, it has been possible to show for the first time that the inhibition of the GEF activity of the endogenous cytohesin blocks the important regulative attachment of leukocytes to the extracellular ligands ICAM-1, which represents a new starting point for the development of more specific immunosuppressive medicaments.

### EXAMPLE 6

### Selection of Aptamers Binding to the Sec7-Domain of Cytohesin-1

Further aptamers against the Sec7 domain of cytohesin-1 were selected in accordance with the following process steps basically in a similar fashion to the procedure described in Example 1.

Selection Conditions

The following buffer was used as binding buffer: 20 mM tris pH 7.5, 100 mM NaCl, 5 mM MgCl₂. The incubation time was 7 minutes at 22° C. The pool used, i.e., the nucleic acid library used as the initial library had the following structure: 5′-GGGATAGGATCCACATCTACGTATTA N30 TTCACTGCAGACTTGACGAAGCTT-3′ (SEQ ID No. 15), where N30 stands for 30 randomised positions with a balanced ratio of the four bases A, C, G and T of 1:1:1:1. As primers for the amplification 5′-GATAATACGACTCACTATA GGG ATA GGA TCC ACA TCT ACG T-3′ (SEQ ID No. 16) was used as the 5′-primer and for the 3′ end: 5′-AAG CTT CGT CAA GTC TGC AGT GAA-3′ (SEQ ID No. 17). The conditions for carrying out the PCR were: 45 sec at 94° C., 60 sec at 50° C. and 90 sec at 72° C. The amplification consisted of 20 PCR cycles for rounds 1-6 and 16 PCR cycles for rounds 7-11. Separation was carried out by washing twice with 125 μl of binding buffer, resuspending in 250 μl of binding buffer, washing twice with 125 μl of binding buffer and then resuspending with 250 μl binding buffer. Elution was carried out using 51 μl of H₂O for 3 min at 98° C. The total selection took place over 11 selection cycles.

Preparation of the Target

The target, i.e., the Sec7 domain of cytohesin-1 was biotinylated and then coupled to streptavidin-coated beads (Magnetic Dynabeads von Dynal). The Sec-7 domain was biotinylated as follows: 100 μg of Sec7 domain was dissolved in PBS, mixed with a tenfold molar excess of Sulfo-NHS-LC-Biotin (obtainable from Pierce) and then incubated for 30 min at 4° C. and then for a further 30 min at room temperature. Excess Sulfo-NHS-LC-Biotin was removed by gel filtration using BioRad Micro Spin Columns 6P. Approximately 70 μg of biotinylated Bio-Sec7 domain was then incubated with 5 mg of Magnetic Dynabeads for 30 min in the overhead shaker. The beads were then washed with PBS/BSA and binding buffer without MgCl₂, equilibrated in binding buffer without MgCl₂ and stored at 4° C. 400 μg of beads was used for each selection cycle.

Analysis of the Enriched Pool

The ³³P-GTP-labelled RNA formed in 50 μl transcription attempts was purified using Qiaquick Nucleotide Removal Kits according to the information given by the manufacturer (Qiagen). The binding studies were carried out for 1 h at 37° C., with a 1 μl aliquot of the purified RNA used in each binding assay.

The result of the binding test is shown in FIG. 12. A total of three pools were studied in relation to their binding behaviour to the target or to the beads used for the selection without target. In this case NC1F Rnd0 denotes the unselected pool, NC1F Rnd6 denotes the pool obtained after 6 selection rounds and NC1F Rnd11 denotes the pool obtained after 11 selection rounds. It can be established that after 11 selection rounds there is considerable enrichment of aptamers specifically binding the target which, although to a lesser extent, can also be observed after 6 selection rounds.

The result of a sequence analysis of the selected aptamers is summarised in the following table.

TABLE
Aptamers selected against the Sec7 domain of cytohesin-1

| | | |
|---|---|---|
| internal designation | selected sequence | SEQ ID No. |

| | | |
|---|---|---|
| NC1F-C11 | ccaccagggataaggtcgaaccctgtcgtc | 18 |
| NC1F-C14 | ccaccagggataaggtcgaaccctgtcgtc | 19 |
| NC1F-C16 | ccaccagggataaggtcgaaccctgtcgtc | 20 |
| NC1F-C17 | ccaccagggataaggtcgaaccctgtcgtc | 21 |
| NC1F-C18 | ccaccagggataaggtcgaaccctgtcgtc | 22 |
| NC1F-C20 | ccaccagggataaggtcgaaccctgtcgtc | 23 |
| NC1F-C21 | ccaccagggataaggtcgaaccctgtcgtc | 24 |
| NC1F-C22 | ccaccagggataaggtcgaaccctgtcgtc | 25 |
| NC1F-C23 | ccaccagggataaggtcgaaccctgtcgtc | 26 |
| NC1F-C25 | ccaccagggataaggtcgaaccctgtcgtc | 27 |
| NC1F-C4 | ccaccagggataaggtcgaaccctgtcgtc | 28 |
| NC1F-C9 | ccaccagggataaggtcgaaccctgtcgtc | 29 |
| NC1F-C10 | atacagtataaccggcatgccgcatcgac | 30 |
| NC1F-C15 | atacacgattccaggccgggaaacgaggac | 31 |
| NC1F-C19 | ataccataacaccgccggcctgtgtacgcg | 32 |
| NC1F-C2 | atacaccagcgcgccgagactaactccgac | 33 |
| NC1F-C3 | ccgaccggccatattacgcgtaatctacc | 34 |
| NC1F-C1 | cgggcccacgcgcgaccgacctcctaacca | 35 |
| NC1F-C12 | accccccactcgacaacccaacgcccgcta | 36 |
| NC1F-C13 | atgcatgcctacgacgcccgccggtcacg | 37 |
| NC1F-C7 | gcgctgatacatcgacctcgcctaacaagg | 38 |
| NC1F-C8 | ggccggcaatgcgtaaacagggcccagaag | 39 |

The afore-mentioned nucleic acids (SEQ ID No. 18-39) can in another embodiment completely or partly comprise at least one of the constant parts of the sequence of the starting library i.e., the constant part of the 5′ end (5′-GGGATAGGATCCACATCTACGTATTA) and/or the 3′ end (TTCACTGCAGACTTGACGAAGCTT-3′).

It is in this connection within the scope of the usual capabilities of the person skilled in the art to shorten these sequences or nucleic acids in order to determine the minimum part of the molecule or sequence required for a binding to the target molecule.

The features of the invention disclosed in the previous description, the claims, the sequence protocol and the drawings can both singly and in arbitrary combination be important for the implementation of the invention in its various embodiments.

1. A nucleic acid, particularly an isolated nucleic acid which is capable of binding to a guanine nucleotide exchange factor for ADP ribosylation factors, and its derivatives.

2. A nucleic acid, particularly an isolated nucleic acid which is capable of binding to a guanine nucleotide exchange factor for ADP ribosylation factors, characterised in that the nucleic acid comprises a sequence selected from the group comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28 SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38 and SEQ ID No. 39 as well as its respective derivatives.

3. The nucleic acid according to claim 1 or claim 2, characterised in that the guanine nucleotide exchange factor belongs to the group of small guanine nucleotide exchange factors for ARF proteins, particularly to the group of guanine nucleotide exchange factors whose molecular weight is around 50 kDa or less.

4. The nucleic acid according to any one of claims 1 to 3, characterised in that the guanine nucleotide exchange factor is not inhibited by Brefeldin A.

5. The nucleic acid according to any one of claims 1 to 4, characterised in that the guanine nucleotide exchange factor is cytohesin-1 or cytohesin-2 and particularly the Sec7 domain of cytohesin-1 or cytohesin-2.

6. The nucleic acid according to any one of claims 1 to 5, characterised in that the guanine nucleotide exchange factor is the Sec7 domain.

7. The nucleic acid according to any one of claims 1 to 6, characterised in that the nucleic acid is selected from the group comprising DNA, RNA, polynucleotides, oligonucleotides, aptamers, aptazymes and intramers.

8. A vector, preferably an expression vector, comprising a nucleic acid according to any one of claims 1 to 7.

9. A cell comprising a nucleic acid according to any one of claims 1 to 7 and/or a vector according to claim 8.

10. The cell according to claim 9, characterised in that the cell is a eukaryotic cell, preferably an animal cell and more preferably a mammalian cell.

11. The cell according to claim 9 or claim 10, characterised in that the cell is selected from the group comprising Saccharomyces cerevisiae and C. elegans.

12. An animal, preferably a transgenic animal, comprising at least one cell according to any one of claims 9 to 11.

13. Use of a nucleic acid according to any one of claims 1 to 7 for the manufacture of a medicament.

14. The use according to claim 13, characterised in that the medicament is for the treatment of diseases selected from the group comprising the metastasis of lymphomas or melanomas, autoimmune diseases, rejection reactions, acute and chronic inflammations, reperfusion damage, transplantation diseases, particularly rejection reactions in organ transplantations and graft-vs-host diseases.

15. The use according to claim 13 or claim 14, characterised in that the medicament influences the β0-2-integrin-mediated adhesion of immune cells.

16. Use of a nucleic acid according to any one of claims 1 to 7 and/or a vector according to claim 8 in gene therapy.

17. Use of a nucleic acid according to any one of claims 1 to 7 for detection of a guanine nucleotide exchange factor.

18. Use of a nucleic acid according to any one of claims 1 to 7 for complex formation with a guanine nucleotide exchange factor.

19. A composition, particularly a pharmaceutical composition, comprising a nucleic acid according to any one of claims 1 to 7, a vector according to claim 8 and/or a cell according to any one of claims 9 to 11 together with a suitable carrier material.

20. An inhibitor for a guanine nucleotide exchange factor, wherein the guanine nucleotide exchange factor belongs to the group of small guanine nucleotide exchange factors for ARF proteins, particularly to the group of guanine nucleotide exchange factors whose molecular weight is around 50 kDa or less.

21. The inhibitor according to claim 20, characterised in that the guanine nucleotide exchange factor is not inhibited by Brefeldin A.

22. The inhibitor according to claim 20 or claim 21, wherein the inhibitor comprises a nucleic acid according to any one of claims 1 to 7 and/or a vector according to claim 8.

23. A method for screening compounds which inhibit the interaction between a guanine nucleotide exchange factor and a nucleic according to claims 1 to 7, especially by a method that is compatible with high-throughput methods and is characterised by the following steps:
a) providing the guanine nucleotide exchange factor and the nucleic acid
b) optionally determining whether an interaction takes place between the guanine nucleotide exchange factor and the β-2-integrin,
c) adding a candidate compound and
d) determining whether an interaction between the guanine nucleotide exchange factor and the nucleic acid is inhibited, preferably by the candidate compound.

24. The method according to claim 23, characterised in that provided as a further step is determining whether the identified compound inhibits the guanine nucleotide exchange function of the guanine nucleotide exchange factor for a monomeric G protein.

25. The method according to claim 23, characterised in that provided as a further step is determining whether the interaction of a guanine nucleotide exchange factor with an integrin, preferably the interaction between the Sec-7 domain of cytohesin-1 and the β-2-integrin subunit, is inhibited.

26. The method according to claim 23, characterised in that provided as a further step is determining whether the interaction of the PH domain with its natural ligands is inhibited, preferably whether the interaction of the PH domain of cytohesin-1 with phosphatidylinositol-3,4,5-trisphosphate is inhibited.

27. The method according to any one of claims 23 to 26, characterised in that the candidate substance is used for the manufacture of a medicament.

28. Use of a guanine nucleotide exchange factor as a target molecule as part of an in vitro selection process.

29. A method for identifying and isolating nucleic acids capable of binding to a target molecule, characterised by the following steps:
incubating the target molecule or a part thereof with a plurality of nucleic acids, preferably a nucleic acid library, wherein the nucleic acids have different sequences,
selecting and isolating those nucleic acids capable of binding to the target molecule or a part thereof,
optionally amplifying the isolated nucleic acids and repeating the first two steps; and
optionally determining the sequence and/or binding specificity of the isolated nucleic acids,
characterised in that the target molecule is a guanine nucleotide exchange factor, preferably one such for ARF.

30. Use of a nucleic acid according to any one of the preceding claims, preferably in a complex with a guanine nucleotide exchange factor, for the rational design of compounds, preferably of inhibitors and more preferably of low-molecular inhibitors.
